SEQUENCE LISTING (22 sequences)

SEQ ID NO: 1 - DNA, 25 bp, Artificial sequence
  primer
tcagccgtcaccgtcagcataacac

SEQ ID NO: 2 - DNA, 25 bp, Artificial sequence
  primer
ccgacaggcgtgcaatgcgcgcagc

SEQ ID NO: 3 - DNA, 30 bp, Artificial sequence
  primer
aaaagaattcgaggcaaaaatgagcaaagt

SEQ ID NO: 4 - DNA, 30 bp, Artificial sequence
  primer
ccccaagcttcatgcaaaaaggggaggcat

SEQ ID NO: 5 - DNA, 29 bp, Artificial sequence
  primer
cggggtaccttctggcgtcaggcagccat

SEQ ID NO: 6 - DNA, 30 bp, Artificial sequence
  primer
acatgcatgccgtctacgcccagcagtttc

SEQ ID NO: 7 - DNA, 28 bp, Artificial sequence
  primer
cggaattcgtatgaagggcgtcagcgcg

SEQ ID NO: 8 - DNA, 29 bp, Artificial sequence
  primer
cggggtaccttcttaagtcacggaattgt

SEQ ID NO: 9 - DNA, 2867 bp, Escherichia coli
  CDS (1)..(2541)
atgagcaaagtcagactcgcaattatcggtaacggtatggtcggccatcgctttatcgaagatcttcttgataaatctgatgcggccaactttgatattaccgttttctgtgaagaaccgcgcatcgcttatgaccgcgtacacctctcgtcttacttctctcaccacaccgccgaagagctgtcgctggtgcgcgaaggcttctacgagaaacacggcatcaaagttctggtcggcgaacgcgctatcaccatcaaccgtcaggagaaggtgattcactccagcgccggacgtaccgttttttatgacaagctgatcatggcaaccggttcctacccgtggatcccgccaatcaaaggttctgatactcaggactgctttgtctatcgcactattgaagacctcaacgccattgaatcctgcgcccgtcgcagcaaacgcggtgccgttgttggtggcggcctgttaggtctggaagccgcaggcgcgctgaaaaacttaggtattgaaacccacgttatcgaatttgcccctatgctgatggcagaacagcttgatcagatgggcggcgagcagctgcgtcgcaaaatcgaaagtatgggcgtgcgcgttcacaccagcaaaaacacccttgagattgtgcaggaaggtgttgaagcgcgtaaaaccatgcgttttgccgacggcagcgaactggaagtcgactttatcgtcttctctaccggtatccgtccgcgcgataagctggcaacccagtgtggtctggacgttgctccgcgtgggggtattgtcattaatgattcctgccagacttccgatccggatatctacgccatcggtgaatgcgcaagctggaacaaccgtgtatttggtctggtagcacctggctacaaaatggcgcaggtcgccgttgaccatattctcggtagcgaaaacgcctttgaaggtgctgaccttagcgccaagctgaaactgctgggcgtagacgtaggcggtattggtgatgcgcacggtcgcacgcctggcgcacgtagctacgtttacctcgacgaaagtaaagagatctacaaacgcctgattgtcagcgaagacaacaaaaccctgctcggtgcggtactggtgggcgataccagcgactacggtaacctgctgcaactggtgctgaacgctatcgaactgccggaaaacccggattccctgatcctgccagcacactcgggtagcggcaagccgtctatcggtgttgataaactgccggacagcgcgcaaatctgctcctgcttcgacgtcaccaaaggtgatctgattgctgccatcaacaaaggctgccacacagttgcggcgctgaaagctgaaaccaaagcgggtactggctgcggtggctgtatcccgctggtcactcaggtactgaacgcggaactggcgaaacagggcatcgaagttaacaacaacctgtgcgaacactttgcttattcgcgtcaggaactgttccatttgatccgcgttgaaggcattaaaaccttcgaagaactgctggcgaaacacggcaaaggctacggttgtgaagtttgtaaaccaaccgtcggttcgctgctggcctcctgctggaacgaatacattctgaagccggaacatactccgctgcaggattctaacgacaacttcctcgctaacatccagaaagacggcacctactcggtgatcccgcgttctccgggcggtgaaatcaccccggaagggctgatggcggtaggtcgtatcgcgcgtgaatttaatctctacaccaagatcactggctcccagcgtctggcgatgtttggcgcacagaaagacgatctgccggagatctggcgtcagctgattgaagccggcttcgaaaccggtcatgcctatgcgaaagcactgcgtatggcgaaaacctgcgtgggtagcacctggtgccgctacggcgttggcgacagcgtcggcctcggcgtggaactggaaaaccgctacaaaggcatccgtacgccgcacaaaatgaagttcggtgtctccggctgtacccgtgaatgttcagaagctcagggtaaagacgtgggtattatcgccactgaaaaaggctggaacctgtatgtttgcggtaacggcggcatgaaaccgcgtcatgcggatctgctggcggcggatatcgatcgcgaaacgctgatcaaatatctcgaccgcttcatgatgttctacatccgtactgccgacaaactgacgcgtaccgcaccgtggttagaaaacctcgaaggcggcatcgattacctgaaagcagtgatcattgacgacaaactggggctgaacgcacatctggaagaagagatggcgcgcctgcgtgaagcggtactgtgtgagtggactgaaacggtcaatacgccgtctgcgcagactcgcttcaaacacttcatcaacagcgacaagcgtgacccgaacgtgcagatggtgccagagcgcgaacagcaccgtccggcaacgccgtatgaacgtatcccagtaactctggtggaggacaacgcatgagccagtggaaagacatctgcaaaatcgatgacatcctgcctgaaaccggcgtctgcgcgctgttaggtgacgagcaggtcgcgattttccgcccgtatcacagcgatcaggtgtttgcgatcagcaacatcgacccgttcttcgagtccagcgtgctgtcacgcggactgattgcggaacaccagggcgagctgtgggtcgccagcccgctgaaaaaacagcgttttcgcttaagcgacggcttgtgcatggaagacgaacagttttccgtcaaacattacgaagcgcgagtgaaagacggcgtggtgcagctgcgcggttaa

SEQ ID NO: 10 - protein, 847 aa, Escherichia coli
MSKVRLAIIGNGMVGHRFIEDLLDKSDAANFDITVFCEEPRIAYDRVHLSSYFSHHTAEELSLVREGFYEKHGIKVLVGERAITINRQEKVIHSSAGRTVFYDKLIMATGSYPWIPPIKGSDTQDCFVYRTIEDLNAIESCARRSKRGAVVGGGLLGLEAAGALKNLGIETHVIEFAPMLMAEQLDQMGGEQLRRKIESMGVRVHTSKNTLEIVQEGVEARKTMRFADGSELEVDFIVFSTGIRPRDKLATQCGLDVAPRGGIVINDSCQTSDPDIYAIGECASWNNRVFGLVAPGYKMAQVAVDHILGSENAFEGADLSAKLKLLGVDVGGIGDAHGRTPGARSYVYLDESKEIYKRLIVSEDNKTLLGAVLVGDTSDYGNLLQLVLNAIELPENPDSLILPAHSGSGKPSIGVDKLPDSAQICSCFDVTKGDLIAAINKGCHTVAALKAETKAGTGCGGCIPLVTQVLNAELAKQGIEVNNNLCEHFAYSRQELFHLIRVEGIKTFEELLAKHGKGYGCEVCKPTVGSLLASCWNEYILKPEHTPLQDSNDNFLANIQKDGTYSVIPRSPGGEITPEGLMAVGRIAREFNLYTKITGSQRLAMFGAQKDDLPEIWRQLIEAGFETGHAYAKALRMAKTCVGSTWCRYGVGDSVGLGVELENRYKGIRTPHKMKFGVSGCTRECSEAQGKDVGIIATEKGWNLYVCGNGGMKPRHADLLAADIDRETLIKYLDRFMMFYIRTADKLTRTAPWLENLEGGIDYLKAVIIDDKLGLNAHLEEEMARLREAVLCEWTETVNTPSAQTRFKHFINSDKRDPNVQMVPEREQHRPATPYERIPVTLVEDNA

SEQ ID NO: 11 - DNA, 327 bp, Escherichia coli
  CDS (1)..(324)
atgagccagtggaaagacatctgcaaaatcgatgacatcctgcctgaaaccggcgtctgcgcgctgttaggtgacgagcaggtcgcgattttccgcccgtatcacagcgatcaggtgtttgcgatcagcaacatcgacccgttcttcgagtccagcgtgctgtcacgcggactgattgcggaacaccagggcgagctgtgggtcgccagcccgctgaaaaaacagcgttttcgcttaagcgacggcttgtgcatggaagacgaacagttttccgtcaaacattacgaagcgcgagtgaaagacggcgtggtgcagctgcgcggttaa

SEQ ID NO: 12 - protein, 108 aa, Escherichia coli
MSQWKDICKIDDILPETGVCALLGDEQVAIFRPYHSDQVFAISNIDPFFESSVLSRGLIAEHQGELWVASPLKKQRFRLSDGLCMEDEQFSVKHYEARVKDGVVQLRG

SEQ ID NO: 13 - DNA, 7320 bp, Escherichia coli
gatcacgcaaaagtagaattgtgcaaataaacggcagggtaatttttgaaggtcagcaacaaaagttgattaattctttgaggaacatgcagttatgcatgctgttgaaaaagaggaagatactgactaactctaaagtggtattttacatgcacttacaattgattaaagacaacattcacagtgtggttatttgttacacataggggcgagcaatgtcatgacagtgtaggtgcggttactgtcgtgaaaaacaataaaaaccgccattgcaacaatggcgcaattcggatgaagcccctatgacaaggataaaaataaacgcacgccgtatcttcagcttattgattccttttttctttttcacttctgttcacgctgaacaaacggctgctcccgcaaaacctgtaactgtggaagcgaagaatgaaacctttgccccgcagcatcccgatcaatatctctcctggaaagccacctcggaacagtcagagcgtgttgacgccctggcggaagatccacggctggtgatcctgtgggcggggtatcccttctcgcgcgattacaacaagccgcgtggacatgcttttgctgtgaccgatgtgcgtgaaaccctgcgtaccggtgcgccgaaaaacgctgaagatggtccgctaccgatggcatgctggagttgtaaaagcccggatgtggcgcgtctgatccagaaagacggcgaagatggctacttccacggtaaatgggcgcgcggcggtccggaaatcgtcaacaacttaggttgtgccgattgccataacaccgcctctccagagttcgccaaaggcaaaccggagttaaccctttcccgtccgtatgcggctcgcgcgatggaagccattggtaaaccttttgagaaagccggacgtttcgaccagcaatcgatggtttgcggtcagtgccatgtggagtattacttcgacggcaaaaacaaagcggttaaattcccgtgggatgacggcatgaaagtcgaaaatatggagcagtattacgacaaaattgccttctctgactggactaactccctgtcgaaaacgccaatgctgaaagcgcagcacccggaatatgaaacctggacagcgggcattcacggtaaaaacaacgtgacctgtatcgactgccatatgccaaaagtgcagaacgccgaaggcaaactctacaccgaccataaaattggtaatccgtttgataacttcgcccagacttgtgcgaactgccatacccaggacaaagctgccttgcaaaaagtggtcgcggaacgtaagcagtcgattaacgacctgaaaatcaaggttgaagatcaactggttcacgctcacttcgaagcgaaagcagcgctggatgcaggcgcgacggaagctgaaatgaagccaattcaggacgatatccgtcatgcccagtggcgctgggatctggcgatcgcttcccacggcattcatatgcacgcaccggaagaaggtttacggatgctcggtacggcgatggataaagcggcggatgcacgcaccaaactggcgcgcctgctggcgaccaaaggcatcacccatgaaatccagatcccggatatctcaaccaaagagaaagcccagcaggccattggcctgaacatggaacaaatcaaggccgagaagcaggacttcatcaaaacggtgatcccgcagtgggaagaacaggcacgtaaaaacggtctgttaagccaataaccccgttccgcctcgcaaggggcggaaaacacaatggagtgaatatgagcgtattacgttcgttattaactgccggggtgctggcgtcgggcctgttgtggagcctgaacggaattaccgctacccctgcggcgcaggcatctgatgatcgctacgaagttacccagcagcgtaacccggatgccgcctgtctggactgtcataaaccagataccgaaggtatgcatggaaaacatgcttccgttatcaacccgaataacaaactgccggtcacctgcaccaactgtcacggccagccatcaccgcaacaccgcgaaggggtgaaagatgtgatgcgctttaacgagccgatgtacaaggttggagagcagaacagcgtctgtatgtcctgtcatctgccggaacagttgcaaaaagcgttctggccgcacgatgtccacgtaaccaaagtggcgtgcgccagctgccattccctgcatccacagcaagatacgatgcagacgttaagtgacaaaggacggattaagatttgcgtcgattgccacagcgatcagcgcaccaatccgaactttaacccggcgtctgttccgttgcttaaggagcagccatgacctggtctcgtcgccagtttctcaccggcgtcggcgtgctggcagccgtcagcggcaccgcagggcgcgtggtggcgaaaacgttgaatatcaatggggtgcgttacggcatggtgcatgacgagtcgttatgcatcggctgtaccgcctgtatggatgcttgtcgggaagtgaacaaggtgccggaaggcgtctcgcgcctgacgattattcgtagcgagccgcagggcgaatttcctgatgtgaaatatcgcttcttccgtaagtcttgccagcactgcgatcatgcgccgtgcgttgacgtctgcccgaccggtgcgtcttttcgcgatgctgccagtgggattgtcgatgttaatccggatctctgcgtcggttgtcagtactgcatcgccgcctgtccgtaccgcgtgcgctttatccatccggtcacgaagacggcggacaaatgcgatttctgccgtaagaccaatttgcaggccggtaagttgcccgcgtgcgttgaagcttgcccgaccaaggcgctgacgtttggcaatctggacgatcccaacagtgagatttcgcaactgctgcgccagaagcccacttaccgctacaagctggcgctgggaaccaaaccgaagctgtaccgcgtaccgtttaaatacggggaggtgagccaatgacgcagacttccgcatttcattttgaatcgctggtgtgggactggccgattgccatctacctgtttttgattggtatttctgctggtctggtgacgctggccgtgctgttacgtcgcttctacccgcaggcgggcggtgcagacagtacgttgctgcgcaccacgctgattgtcgggccgggcgcggtgatcctcggtctgttgatcctcgtcttccacctgacaagaccgtggaccttctggaagctgatgttccactacagttttacctcggtgatgtcgatgggggtgatgctgtttcagctctacatggtggtgctggtgctgtggctggcgaaaatctttgaacatgatttgcttgccctgcaacaacgctggttgccgaagctggggatcgtgcaaaaggttctgagcctcgtgacgcccgttcatcgcggactggaaacattgatgctggtgctggcggtgttgttgggggcttataccggctttctgctgtcggcgctgaaatcgtatccgttcctcaataacccgatcctgccggtgctgttcctcttctccggcatctcgtccggtgcggcggtggcgctgatcgccatgcggatacgccaacgcagtaacccgcattccacggaagcgcagtttgtacaccgtatggaaatccctgtggtatggggtgaaatcttcctgctggtggcgttttttgtcggtctggcgctgggcgatgacggtaaagttcgtgcgctggtggcggcattaggtggcggtttctggacgtggtggttctggcttggtgtcgccgggctggggctgattgtgccaatgctgctcaaaccgtgggtcaatcgcagttccggcattcctgccgtgctggcggcgtgtggggccagtctggtcggcgtgttgatgctgcgctttttcattctctacgccggacagttgacggtggcgtaagccagaaaagaggtggtttctggacgtattccttcctgaagtcggttttctggcgttgttgttaagtctcggggtcaacgtgttgaccccgttgacggccttcgcgggagtgcggttgcgctggcctgccatgatgcgactcacttgcatcggcattctggcgcagttcgccgtcctgctgctcgcctttgcggtactgacgtattgttttctcatcagcgatttctcggtcatttatgtcgcgcaacatagctatagcctgctgtcgtgggaactcaagctggcagcggtgtggggcggccatgaaggttcgctgctgctttgggtgctgctgctttccgcctggagcgcgctgtttgcctggcattatcggcagcaaaccgatccgctatttccgctgacgttagccgttttatctctcatgctcgccgcactgctactgtttgtggtgctgtggtccgatcccttcgtgcggatatttccaccagcaatcgaaggccgcgatctcaatccgatgctgcaacatcccggtcttatctttcatccaccgctgctttaccttggctatggcggtttgatggtagcggcgagcgtggcgctggcgagtctactgcgcggcgagtttgatggtgcctgcgcccgaatttcgtggcgctgggcgttacctggctggagtgcattaacggcggggatcatcctcggttcctggtgggcctactgcgaactcggcctggggcggctggtggttctgggatccgtggaaaacgcctctttattaccctggctttctgccactgcgctgctgcacagtttgtccctgacacgccagcgggggattttttgccactggtcgctgttgctggcgatagttactctgatgctgtcgctgctgggcaccttaattgtccgttctggcattctggtttcggttcatgcgttcgcgctggataacgtccgcgccgtgccgttgttcagcctgtttgcactgattagccttgcgtctctggctctgtatggctggcgagcgcgggacggtggcccggcggtgcatttttcggggttatcgcgggaaatgttaatcctcgctacgctgttgctgttttgcgcagtgctactgatcgtgctggtgggaacgctttatccgatgatttacggcctgctgggctggggacgcctctccgttggcgcgccgtattttaaccgcgcgacgttaccgtttggtctgttgatgctggtggtgattgtgctggcgacgtttgtctctggcaaacgcgtgcagcttccggcgctggtagctcatgcgggcgtgctgttatttgccgctggggtcgtggtctccagcgtcagccgtcaggagatcagcctgaatttacagccgggtcagcaggtgacgctggcaggatacaccttccgttttgagtgcctcgatctacaagccaaagcgaattacaccagcgaaaaagcgatagtggcactgtttgaccatcagcaacgcattggtgaactgacgccggagcgggctttttatgaagcacgccgtcaacaaatgatggaaccgtcaattcgctggaacggcatccatgactggtatgcggtcatgggggagaaaactgggccggatcgttacgcttttcgtttgtatgtacaaagcggtgtgcgctggatctgggggggaggattgttgatgattgcgggcgcattgttaagcggatggcgggggaagaagcgcgatgaataaagggcttctcacgctactgctgttatttacctgttttgcgcacgctcaggtcgtagacacctggcaattcgccaatccgcaacaacagcaacaggcgttaaatattgccagccagttacgttgtccgcagtgccagaatcaaaacttactggaatccaacgcgccggtggctgtcagtatgcgccatcaggtttacagcatggtggcggaggggaagaacgaagtcgaaatcattggctggatgaccgaacgctacggagattttgttcgctataacccaccgttaacgggtcagacgctggtgttatgggcgctgccagtggtgttgttactgctgatggcactgatcctctggcgagtgagggcgaagcgatgaaacagcccaaaataccggtgaaaatgctgacaacactcacgatattgatggtatttctctgtgtcggcagttatctgttaagcccaaaatggcaggcggtacgtgcggagtatcagcgtcagcgcgatccgctacatcagtttgccagccagcaaaccccggaagcgcagcttcaggcattgcaggataaaatccgcgctaatccacaaaacagcgaacagtgggcgttactgggcgagtactatctgtggcaaaacgattacagcaattcgttgctggcgtaccgtcaggcgttgcaactacgtggtgagaacgctgaactgtatgcggcgctggcgacggtgctttattaccaggccagccagcatatgaccgcccagactcgcgcaatgatcgacaaagccctcgcgctggacagtaatgaaatcaccgccctgatgctgctggcttccgatgcgtttatgcaggcgaactacgcgcaagccatcgaactatggcaaaaagtgatggatctcaactcaccgcgagttaaccgaacacagctggttgagtcgattaatatggcgaaattgttgcagcggagattggattaatcatctggtgatgaagattatgtcattgagctgcataaaaaataatcgaatgaacatatgccaaaaataatcactaatcagtattattgcagattaaacaaataaaaatctttccataacaaatggttattcattaatcctgccataaactcgcgttattatgcattaatgcagcgaaaagctctgttgttaaagggttgcgcaacataccgcgcaaatgatactgatcataagcgttaaaaaaatctacaaaccaacgcaacacaattcatgccctggcagtatgtcacgttctcgcgtttctgaacggggaacggcgctccattgaggaagtcattcatatgaaaaatataaaattcagcctggcctggcagattctgtttgctatggtgctgggcattctcctgggaagctacctgcactaccatagcgacagccgcgactggctggtcgtcaatttgctctctccggcgggtgatatcttcatccatctgattaaaatgattgttgtgccgattgtgatctccacgctggtggtgggtatcgcgggtgttggtgatgccaaacagctcgggcgtattggcgcgaaaaccattatctacttcgaggtgatcaccaccgtcgccatcattttggggatcc

SEQ ID NO: 14 - protein, 450 aa, Escherichia coli
MTRIKINARRIFSLLIPFFFFTSVHAEQTAAPAKPVTVEAKNETFAPQHPDQYLSWKATSEQSERVDALAEDPRLVILWAGYPFSRDYNKPRGHAFAVTDVRETLRTGAPKNAEDGPLPMACWSCKSPDVARLIQKDGEDGYFHGKWARGGPEIVNNLGCADCHNTASPEFAKGKPELTLSRPYAARAMEAIGKPFEKAGRFDQQSMVCGQCHVEYYFDGKNKAVKFPWDDGMKVENMEQYYDKIAFSDWTNSLSKTPMLKAQHPEYETWTAGIHGKNNVTCIDCHMPKVQNAEGKLYTDHKIGNPFDNFAQTCANCHTQDKAALQKVVAERKQSINDLKIKVEDQLVHAHFEAKAALDAGATEAEMKPIQDDIRHAQWRWDLAIASHGIHMHAPEEGLRMLGTAMDKAADARTKLARLLATKGITHEIQIPDISTKEKAQQAIGLNMEQ

SEQ ID NO: 15 - protein, 160 aa, Escherichia coli
MSVLRSLLTAGVLASGLLWSLNGITATPAAQASDDRYEVTQQRNPDAACLDCHKPDTEGMHGKHASVINPNNKLPVTCTNCHGQPSPQHREGVKDVMRFNEPMYKVGEQNSVCMSCHLPEQLQKAFWPHDVHVTKVACASCHSLHPQQDTMQTLSDKGRI

SEQ ID NO: 16 - protein, 218 aa, Escherichia coli
MTWSRRQFLTGVGVLAAVSGTAGRVVAKTLNINGVRYGMVHDESLCIGCTACMDACREVNKVPEGVSRLTIIRSEPQGEFPDVKYRFFRKSCQHCDHAPCVDVCPTGASFRDAASGIVDVNPDLCVGCQYCIAACPYRVRFIHPVTKTADKCDFCRKTNLQAGKLPACVEACPTKALTFGNLDDPNSEISQLLRQKPTYRYKLALGTKPKLYRVPFKY

SEQ ID NO: 17 - protein, 318 aa, Escherichia coli
MTQTSAFHFESLVWDWPIAIYLFLIGISAGLVTLAVLLRRFYPQAGGADSTLLRTTLIVGPGAVILGLLILVFHLTRPWTFWKLMFHYSFTSVMSMGVMLFQLYMVVLVLWLAKIFEHDLLALQQRWLPKLGIVQKVLSLVTPVHRGLETLMLVLAVLLGAYTGFLLSALKSYPFLNNPILPVLFLFSGISSGAAVALIAMRIRQRSNPHSTEAQFVHRMEIPVVWGEIFLLVAFFVGLALGDDGKVRALVAALGGGFWTWWFWLGVAGLGLIVPMLLKPWVNRSSGIPAVLAACGASLVGVLMLRFFILYAGQLTVA

SEQ ID NO: 18 - protein, 552 aa, Escherichia coli
MLTPLTAFAGVRLRWPAMMRLTCIGILAQFAVLLLAFAVLTYCFLISDFSVIYVAQHSYSLLSWELKLAAVWGGHEGSLLLWVLLLSAWSALFAWHYRQQTDPLFPLTLAVLSLMLAALLLFVVLWSDPFVRIFPPAIEGRDLNPMLQHPGLIFHPPLLYLGYGGLMVAASVALASLLRGEFDGACARISWRWALPGWSALTAGIILGSWWAYCELGLGRLVVLGSVENASLLPWLSATALLHSLSLTRQRGIFCHWSLLLAIVTLMLSLLGTLIVRSGILVSVHAFALDNVRAVPLFSLFALISLASLALYGWRARDGGPAVHFSGLSREMLILATLLLFCAVLLIVLVGTLYPMIYGLLGWGRLSVGAPYFNRATLPFGLLMLVVIVLATFVSGKRVQLPALVAHAGVLLFAAGVVVSSVSRQEISLNLQPGQQVTLAGYTFRFECLDLQAKANYTSEKAIVALFDHQQRIGELTPERAFYEARRQQMMEPSIRWNGIHDWYAVMGEKTGPDRYAFRLYVQSGVRWIWGGGLLMIAGALLSGWRGKKRDE

SEQ ID NO: 19 - protein, 127 aa, Escherichia coli
MNKGLLTLLLLFTCFAHAQVVDTWQFANPQQQQQALNIASQLRCPQCQNQNLLESNAPVAVSMRHQVYSMVAEGKNEVEIIGWMTERYGDFVRYNPPLTGQTLVLWALPVVLLLLMALILWRVRAKR

SEQ ID NO: 20 - protein, 198 aa, Escherichia coli
MKQPKIPVKMLTTLTILMVFLCVGSYLLSPKWQAVRAEYQRQRDPLHQFASQQTPEAQLQALQDKIRANPQNSEQWALLGEYYLWQNDYSNSLLAYRQALQLRGENAELYAALATVLYYQASQHMTAQTRAMIDKALALDSNEITALMLLASDAFMQANYAQAIELWQKVMDLNSPRVNRTQLVESINMAKLLQRRLD

SEQ ID NO: 21 - DNA, 1686 bp, Escherichia coli
  CDS (1)..(1683)
atgacaacaaccaccggaagtgcccggccagcacgtgccgccaggaagcctaagcccgaaggccaatggaaaatcgacggcaccgagccgcttaaccatgccgaggaaattaagcaagaagaacccgcttttgctgtcaagcagcgggtcattgatatttactccaagcagggtttttcttccattgcaccggatgacattgccccacgctttaagtggttgggcatttacacccagcgtaagcaggatctgggcggtgaactgaccggtcagcttcctgatgatgagctgcaggatgagtacttcatgatgcgtgtgcgttttgatggcggactggcttcccctgagcgcctgcgtgccgtgggtgaaatttctagggattatgctcgttccaccgcggacttcaccgaccgccagaacattcagctgcactggattcgtattgaagatgtgcctgcgatctgggagaagctagaaaccgtcggactgtccaccatgcttggttgcggtgacgttccacgtgttatcttgggctccccagtttctggcgtagctgctgaagagctgatcgatgccaccccggctatcgatgcgattcgtgagcgctacctagacaaggaagagttccacaaccttcctcgtaagtttaagactgctatcactggcaaccagcgccaggatgttacccacgaaatccaggacgtttccttcgttccttcgattcacccagaattcggcccaggatttgagtgctttgtgggcggtggcctgtccaccaacccaatgcttgctcagccacttggttcttggattccacttgatgaggttccagaagtgtgggctggcgtcgccggaattttccgcgactacggcttccgacgcctgcgtaaccgtgctcgcctcaagttcttggtggcacagtggggtattgagaagttccgtgaagttcttgagaccgaatacctcgagcgcaagctgatcgatggcccagttgttaccaccaaccctggctaccgtgaccacattggcattcacccacaaaaggacggcaagttctacctcggtgtgaagccaaccgttggacacaccaccggtgagcagctcattgccattgctgatgttgcagaaaagcacggcatcaccaggattcgtaccacggcggaaaaggaactgctcttcctcgatattgagagaaagaaccttactaccgttgcacgcgacctggatgaaatcggactgtactcttcaccttccgagttccgccgcggcatcatttcctgcaccggcttggagttctgcaagcttgcgcacgcaaccaccaagtcacgagcaattgagcttgtcgacgaactggaagagcgcctcggcgatttggatgttcccatcaagattgcactgaacggttgccctaactcttgtgcacgcacccaggtttccgacatcggattcaagggacagaccgtcactgatgctgacggcaaccgcgttgaaggtttccaggttcacctgggcggttccatgaacttggatccaaacttcggacgcaagctcaagggccacaaggttattgccgatgaagtgggagagtacgtcactcgcgttgttacccacttcaaggaacagcgccacgaggacgagcacttccgcgattgggtccagcgggccgctgaggaagatttggtgtga

SEQ ID NO: 22 - protein, 561 aa, Escherichia coli
MTTTTGSARPARAARKPKPEGQWKIDGTEPLNHAEEIKQEEPAFAVKQRVIDIYSKQGFSSIAPDDIAPRFKWLGIYTQRKQDLGGELTGQLPDDELQDEYFMMRVRFDGGLASPERLRAVGEISRDYARSTADFTDRQNIQLHWIRIEDVPAIWEKLETVGLSTMLGCGDVPRVILGSPVSGVAAEELIDATPAIDAIRERYLDKEEFHNLPRKFKTAITGNQRQDVTHEIQDVSFVPSIHPEFGPGFECFVGGGLSTNPMLAQPLGSWIPLDEVPEVWAGVAGIFRDYGFRRLRNRARLKFLVAQWGIEKFREVLETEYLERKLIDGPVVTTNPGYRDHIGIHPQKDGKFYLGVKPTVGHTTGEQLIAIADVAEKHGITRIRTTAEKELLFLDIERKNLTTVARDLDEIGLYSSPSEFRRGIISCTGLEFCKLAHATTKSRAIELVDELEERLGDLDVPIKIALNGCPNSCARTQVSDIGFKGQTVTDADGNRVEGFQVHLGGSMNLDPNFGRKLKGHKVIADEVGEYVTRVVTHFKEQRHEDEHFRDWVQRAAEEDLV

(19) United States
(12) Patent Application Publication
(10) Pub. No.: US 2006/0057118 A1
(43) Pub. Date: Mar. 16, 2006

(54) Feed composition for ruminants

(76) Inventors: Yasuhiko Toride, Kawasaki-shi (JP); Junichi Takahashi, Obihiro-shi (JP); Nahoko Isogai, Kawasaki-shi (JP); Yoko Kuwabara, Kawasaki-shi (JP)

Correspondence Address: AJINOMOTO CORPORATE SERVICES LLC, 1120 CONNECTICUT AVENUE, N.W., SUITE 1010, WASHINGTON, DC, 20036, US

(21) Appl. No.: 11/210,756

(22) Filed: Aug. 25, 2005

(30) Foreign Application Priority Data
Aug. 25, 2004 (JP) 2004-245705

(51) Int. Cl. A61K 35/74; A23K 1/165

(52) U.S. Cl. 424/93.4; 424/442

(57) ABSTRACT

A feed composition for ruminants comprising a microbe having nitrite reductase activity, wherein said microbe has been modified so that intracellular nitrite reductase activity is enhanced is disclosed. A feed composition for ruminants comprising a microbe having nitrite reductase activity, wherein said microbe is selected from the group consisting of intestinal bacteria, coryneform bacteria, Bacillus subtilis, bacteria of the genus Methylophilus, Actinomyces, ruminal bacteria, and a combination thereof is disclosed. A method of improving the growth of a ruminant by administering the feed composition as described above to said ruminant and a method of preventing nitrite intoxication by administering the feed composition as described above to a ruminant are disclosed.

### BACKGROUND OF THE INVENTION

2. Field of the Invention

A methane production reducer and a feed composition for ruminants, and more specifically, to a methane production reducer for ruminants characterized by comprising a microbe-derived nitrite reductase as an active ingredient, and to a feed composition for ruminants characterized by comprising this methane production reducer is disclosed.

2. Description of the Related Art

The methane produced in the rumen of a ruminant not only constitutes inefficient use of the energy in feed, but is also a greenhouse gas contributing to global warming. Thus, it is highly important to reduce methane production.

Fermentation is conducted in the rumen by a variety of microbes, resulting in production various metabolites. One of these, methane, is thought to be produced by methane producing bacteria. The methane producing bacteria in the rumen are hydrogen-assimilating bacteria that utilize hydrogen to reduce carbon dioxide, generating methane. Accordingly, the presence of a more powerful reduction reaction inhibits methane production (Japanese Patent Application Publication No. 2003-88301, International Patent Application Publication No. WO96/39860).

The administration of ionophores such as monensin and iberin to ruminants is a known method of inhibiting methane production in the rumen. The supplying of cysteine (Japanese Patent Application Publication No. Heisei 7-322828) and the supplying of fiunaric acid (Japanese Patent Application Publication No. Heisei 11-46694) are known methods that have received attention in the adjustment of reduction capability in the rumen.

It is also known that reduction of nitrates via nitrites and hydroxyamines to ammonia decreases the production of methane in the rumen. However, since nitrite reduction activity is rate-determining in the rumen (Iwamoto M., Asanuma N., Anim Sci J, 1972, 70: 471-478), it is necessary to rapidly reduce nitrites to inhibit methane production. Furthermore, in recent years, the nitrate content of feeds has increased due to the cultivation of forage with nitrogen fertilizer, which creates the problem of large amounts of nitrites accumulation in the rumen.

The toxicity of nitrites accumulating in the rumen is known to decrease the activity of microbes in the rumen (ruminal bacteria) and cause nitrite intoxication in animals. It has become clear that the addition of nitrates to mixed culture systems of ruminal bacteria in an attempt to inhibit the production of methane in the rumen due to nitrate reduction results in an increase in the number of microbes capable of reducing nitrates that are constantly present in the rumen (Iwamoto M., Asanuma N., and Hino T., Anaerobe, 2002, 8, 209-215). Furthermore, Bacterial composition to reduce the toxic effects of high nitrate consumption in livestock, including Propionibacterium acidipropionici, which has the ability to reduce nitrites, and methane production inhibitors, including microbes derived from sheep's milk such as lactobacilli and yeast, have been developed (U.S. Pat. No. 6,120,810, Japanese Patent Application Publication No. 2003-88301).

Microbes such as Escherichia coli (E. coli) are known to have nitrite reductases (encoded by nirBD, nrfABCDEFG, Cole, J., FEMS Microbiology Letters Volume 136, February, 1996, Issue 1, Henian Wang and Robert P. Gunsalus, Journal of Bacteriology, October, 2000, p. 5813-5822, Vol. 182, No. 20), but a feed composition containing E. coli or any other microbe enhancing nitrite acid reducing capability has not been previously developed.

### SUMMARY OF THE INVENTION

A methane production reducer and a feed composition which reduce the production of methane in the rumen of a ruminant, enhance feed efficiency, and/or prevent nitrite intoxication are disclosed. Furthermore, a method of administering the methane production reducer or the feed composition to ruminants is disclosed.

The present inventors discovered that the administration of a microbe-derived nitrite reductase into the rumen fluid of a ruminant, or oral administration of the same, enhanced nitrite reduction activity and reduced methane production in the rumen.

An embodiment of the present invention includes a feed composition for ruminants comprising a microbe having nitrite reductase activity, wherein said microbe has been modified so that intracellular nitrite reductase activity is enhanced.

An embodiment of the present invention includes the feed composition as described above, wherein said microbe is selected from the group consisting of intestinal bacteria, coryneform bacteria, Bacillus bacterium, bacteria of the genus Methylophilus, Actinomyces, ruminal bacteria, and a combination thereof.

An embodiment of the present invention includes the feed composition as described above, wherein said microbe is Escherichia bacterium.

An embodiment of the present invention includes the feed composition as described above, wherein said nitrite reductase comprises a protein selected from the group consisting of:
(A) a protein selected from the group consisting of SEQ ID NO: 10, 12, 14 to 20, 22, and combination thereof; and
(B) a protein selected from the group consisting of SEQ ID NO: 10, 12, 14 to 20, 22, and combination thereof, except that 1 to 30 amino acids have been substituted, deleted, inserted, or added, and wherein said protein has nitrite reductase activity.

An embodiment of the present invention includes the feed composition as described above, wherein said microbe is characterized by enhanced expression of a gene coding for nitrite reductase.

An embodiment of the present invention includes the feed composition as described above, wherein said gene coding for nitrite reductase is derived from a bacterium of the genus Escherichia, a coryneform bacterium, or a bacterium of the genus Bacillus.

An embodiment of the present invention includes the feed composition as described above, wherein said gene coding for said nitrite reductase comprises a nucleotide sequence selected from the group consisting of:
(A) a nucleotide sequence selected from the group consisting of SEQ ID NO: 9, 13, and 21; and
(B) a nucleotide sequence which is able to hybrid under stringent conditions with the nucleotide sequence selected from the group consisting of SEQ ID NO: 9, 13, and 21, or with a probe prepared from one of said nucleotide sequences, wherein said gene codes for a protein having nitrite reductase activity.

An embodiment of the present invention includes a feed composition for ruminants comprising a microbe having nitrite reductase activity, wherein said microbe is selected from the group consisting of intestinal bacteria, coryneform bacteria, Bacillus subtilis, bacteria of the genus Methylophilus, Actinomyces, ruminal bacteria, and a combination thereof.

An embodiment of the present invention includes the feed composition as described above, wherein said nitrite reductase comprises a protein selected from the group consisting of:
(A) a protein selected from the group consisting of SEQ ID NO: 10, 12, 14 to 20, 22, and combination thereof; and
(B) a protein selected from the group consisting of SEQ ID NO: 10, 12, 14 to 20, 22, and combination thereof, except that 1 to 30 amino acids have been substituted, deleted, inserted, or added, and wherein said protein has nitrite reductase activity.

An embodiment of the present invention includes a method of improving the growth of a ruminant by administering the feed composition as described above to said ruminant.

An embodiment of the present invention includes a method of preventing nitrite intoxication by administering the feed composition as described above to a ruminant.

The methane production reducer and feed composition reduce the production of methane in the rumen of a ruminant and enhance the energy efficiency of feed. Furthermore, by reducing the production of methane, a greenhouse gas, it contributes to the solution of environmental issues such as global warming. Still further, the methane production reducer of the present invention is useful in preventing the nitrite intoxication of ruminants.

### BRIEF DESCRIPTION OF THE DRAWINGS

FIG. 1 shows profiles of in vitro cumulative CH₄ production in cultures of mixed ruminal bacteria treated with nitrate.

FIG. 2 shows profiles of in vitro cumulative CH₄ production in cultures of mixed ruminal bacteria treated with nitrite.

FIG. 3 shows profiles of in vitro cumulative CO₂ production in cultures of mixed ruminal bacteria treated with nitrate.

FIG. 4 shows profiles of in vitro cumulative CO₂ production in cultures of mixed ruminal bacteria treated with nitrite.

FIG. 5 shows profiles of ruminal nitrite accumulation in in vitro continuous incubation of mixed ruminal bacteria treated with nitrate.

FIG. 6 shows profiles of ruminal nitrite accumulation in in vitro continuous incubation of mixed ruminal bacteria treated with nitrite.

### DETAILED DESCRIPTION OF THE INVENTION

The present invention is described in detail below.

The methane production reducer of the present invention is characterized by comprising a microbe-derived nitrite reductase as active ingredient and by enhancing nitrite reduction activity in the rumen. The ruminant referred to in the present invention is a mammal belonging to the suborder of ruminants of the Artiodactyla, having a stomach divided into three or four compartments, and ruminating food. Examples are cattle and sheep.

The nitrite reduction activity referred to in the present invention means activity reducing nitrites that is catalyzed by nitrite reductase. “Enhancing nitrite reduction activity” means that the administration of a methane production reducer comprising nitrite reductase derived from a microbe increases nitrite reduction activity in the rumen relative to when no such reducer is administered. It suffices for nitrites in the rumen to be reduced to ammonia to a greater degree than when no such reducer is administered. Nitrite reduction activity, when given per hour per volume (L) of rumen, is desirably greater than or equal to 0.1 mM/h/L, preferably greater than or equal to 0.2 mM/h/L.

Nitrite reductase is an enzyme that catalyzes a nitrite reduction reaction. The nitrite reductase employed as an active ingredient in the present invention may either be derived from microbes originally present in the rumen, or derived from microbes not present in the rumen. It need only be a microbe-derived enzyme having nitrite reduction activity that does not greatly compromise the natural balance of the various microbe groups living symbiotically in the digestive system of a ruminant when administered.

One or more enzyme that is derived from, but not limited to, an intestinal bacterium such as those of microbes of the genera Escherichia, Salmonella, Shigella, and Enterobacter; a coryneform bacterium such as Corynebacterium glutanicum; a bacillus such as Bacillus subtilis; a C1 compound-assimilating bacterium such as those of the genus Methylophylus, genus Achromobacter, genus Pseudomonas, genus Protaminobacter, genus Methylomonas, genus Methylobacillus (Patent Application Publication No. Heisei 4-91793), and genus Bacillus (Patent Application Publication No. Heisei 3-505284); Actinomyces such as Streptomyces coelicolor; a ruminal bacterium such as Pyrobaculum aerophilum, Selenomonas ruminantium, Veillonella parvula, and Wolnella succinogenes; may be employed as the nitrite reducing enzyme of the present invention.

Enzymes derived from intestinal bacterium includes, but not limited to, the enzymes from the bacterium belonging to the genera Enterobacter, Erwinia, Escherichia, Klebsiella, Pantoea, Providencia, Salmonella, Serratia, Shigella, Morganella etc. Specifically, those classified into the Enterobacteriaceae according to the taxonomy used in the NCBI (National Center for Biotechnology Information) database can be used. (Refer to NCBI Home page) A bacterium belonging to the genus of Escherichia was used in the following examples.

Enzymes derived from the bacterium belonging to the genus of Escherichia particularly nontoxic strains K and B (for example, strain W3110 of strain K-12, which is a derivative of the K strain and strain MG1655) reported in Neidhardt et al. (Neidhardt, F. C. et al., Escherichia coli and Salmonella Typhimurium, American Society for Microbiology, Washington D.C., 1208, Table 1), can be utilized. Examples of wild-type strains of Escherichia coli include, but are not limited to, the K12 strain and derivatives thereof, Escherichia coli MG1655 strain (ATCC No. 47076), and W3110 strain (ATCC No. 27325). These strains are available from the American Type Culture Collection (ATCC, Address: P.O. Box 1549, Manassas, Va. 20108, United States of America).

The term “Bacillus bacterium” means that the bacterium classified as the genus Bacillus according to the classification known to a person skilled in the art of microbiology.

The enzyme from Bacillus bacterium include, but are not limited to, the Bacillus subtilis 168 Marburg strain (ATCC 6051), Bacillus subtilis PY79 strain (Plasmid, 1984, 12, 1-9) and so forth, and examples of Bacillus amyloliquefaciens include, but are not limited to, Bacillus amyloliquefaciens T strain (ATCC 23842), Bacillus amyloliquefaciens N strain (ATCC 23845) and so forth.

Examples of coryneform bacteria are microbes belonging to the genus Corynebacterium, microbes belonging to the genus Brevibacterium, and microbes belonging to the genus Arthrobacter (Int. J. Syst. Bacteriol., 41, 255 (1991)). Of these, those belonging to the genera Corynebacterium and Brevibacterium can be used. Examples are enzymes derived from microbes belonging to Corynebacterium glutamicum(ATCC13032 ATCC13869), Brevibacteriumflavum(ATCC13826,14067), Brevibacterium ammoniagenes(ATCC6871,6872), and Brevibacterium lactofermentum(ATCC13869).

Example of ruminal bacteria are microbes belonging to Anaerovibrio, Butyrivibrio Bacteroides, Clostridium, Eubacterium, Lachnospire, Megasparea, Metahnobrevibacter, Methanosarcina, Ruminococcus, Selenomonas Selenomonas Streptococcus, Succinivibrio, Wolinella (nutritional physiology of ruminant;Rural Culture Association 1998).

Enzymes derived from, for example, AS1 strain (NCIMB10515) of Methylophilus methylotrophus, and Bacillus methanolicus, are examples of those derived from C1 compound-assimilating microbes.

The nitrite reductase activity of these enzymes can be measured and confirmed by observing the reduction of nitrites when the enzymes are added to a suitable reaction or bacterial culture system. Furthermore, the activity of those employing ferredoxin as an electron donor (EC 1.7.2.2, EC 1.7.7.1) can be measured by the method of Okunuki et al. (1970, Cytochrome p. 195-207) for EC 1.7.2.2 and the method of Ida et al. (1976, Tanpakushitsu, Kakusan, Koso, special edition, p. 349-355) for EC 1.7.7.1. The activity of those employing NAD(P)H as electron donor (EC 1.7.1.4) can be measured by the method of N. R. Harbourne (Molecular Microbiology, 6, 2805 (1992)).

A nitrite reductase that has been registered in a database (Swiss-Prot, EMBL) may be employed in the present invention.

For example, the following enzymes that have been registered as EC 1.7.1.4 nitrite reductases may be employed; however, the present invention is not limited thereto.
1. sp: NASD_{—}BACSU [P42435] Nitrite reductase [NAD(P)H] (derived from Bacillus subtilis).
2. sp: NASE_{—}BACSU [P42436] Assimilatory nitrate reductase [NAD(P)H] small subunit (derived from Bacillus subtilis).
3. sp: NASB_{—}BACSU Assimilatory nitrate reductase electron transfer subunit.
4. sp: NIRB_{—}ECOLI [P08201] Nitrate reductase [NAD(P)H] large subunit (derived from E. coli) (SEQ ID NO: 10).
5. sp: NIRD_{—}ECOLI [P23675] Nitrite reductase [NAD(P)H] small subunit (derived from E. coli) (SEQ ID NO: 12)

For example, the following enzymes, categorized as EC 1.7.2.2 nitrite reductases, may be employed.
1. sp: NRFA_{—}ECOLI [P32050] Cytochrome c-552 precursor (Ammonia-forming cytochrome c nitrite reductase) (Cytochrome c nitrite reductase) (derived from E. coli) (SEQ ID NO: 14).
2. prf: 2001439E nrfB protein—(derived from E. coli) (SEQ ID NO: 15)
3. prf: 1924370B NrfC protein—(derived from E. coli) (SEQ ID NO: 16)
4. prf: 1924370C NrfD protein—(derived from E. coli) (SEQ ID NO: 17)
5. prf: 2001439EL nrfE protein—(derived from E. coli) (SEQ ID NO: 18)
6. sp: NRFG_{—}ECOLI [P32711] Formate-dependent nitrite reductase complex nrfF subunit precursor (derived from E. coli) (SEQ ID NO: 19)
7. sp: NRFG_{—}ECOLI [P32712] Formate-dependent nitrite reductase complex nrfG subunit (derived from E. coli) (SEQ ID NO: 20)

For example, the following enzymes, categorized as EC 1.7.7.1 nitrite reductases, may be employed.
1. NP_{—}602008 putative nitrite reductase (derived from Corynebacterium glutamicum) (SEQ ID NO: 22)
2. BAC19454 putative ferredoxin-nitrite reductase (derived from Corynebacterium efficies)
3. CAC33947 putative nitrite sulphite reductase (derived from Streptomyces coelicolor)

Many nitrite reductases have a structure comprising multiple subunits. However, some of the subunits may be missing so long as nitrite reduction activity is enhanced in the rumen. To the extent that the nitrite reductase activity of the protein is not lost, the nitrite reductase of the present invention may be anything coding for a nitrite reductase comprising one or several amino acid substitutions, deletions, insertions, additions, or inversion at one or several sites. Here, the term “several” varies with the type and position of the amino acid residues within the three-dimensional structure of the protein, but specifically refers to from 2 to 30, preferably from 2 to 20, and more preferably from 2 to 10 amino acids.

The nitrite reductase of the present invention includes not only enzymes derived from the above-described microbes inherently having nitrite reducing activity, but also enzymes derived from microbes which inherently have no nitrite reductase activity but have acquired ability to reduce nitrites through an introduction of a gene coding for nitrite reductase. Furthermore, the nitrite reductases of the present invention may be derived from wild-type strains of microbes having nitrite reduction capability, but are desirably derived from microbes characterized by having been modified so that intracellular nitrite reductase activity is enhanced.

In the present invention, the phrase “modified so that intracellular nitrite reductase activity is enhanced” means modified to heighten the nitrite reductase activity per cell. This also includes the case where nitrite reductase activity has increased beyond that of the unmodified strain, such as a wild-type strain to the degree of not less than 1.5-fold, not less than 2-fold, and not less than 3-fold of a wild-type or non-modified strain. For example, this corresponds to the case where the number of molecules of nitrite reductase per cell has been increased and the case where the activity per nitrite reductase molecule has been increased. Examples of wild-type strains for comparison are E. coli MG1655 and Bacillus subtilis 168.

Various methods are known for increasing nitrite reductase activity. One method of enhancing enzymatic activity is to increase the expression level of the gene coding for the enzyme. Known methods include the method of increasing the number of the gene copies in extrachromosomal DNA such as plasmids (see U.S. Pat. No. 5,830,716), the method of increasing the number of the gene copies on the chromosomes, the method of introducing a mutation into the promoter of the gene coding for the enzyme to increase activity (see International Publication WO 00/18935), and the method of substituting the promoter with a stronger promoter. Nitrite reductase activity can also be enhanced by introducing a mutation into the coding region of the gene to heighten the specific activity of the nitrite reductase (see Cole J., FEMS Microbiology Letters Volume 136, February 1996, Issue 1). Here, the gene employed may be either a gene coding for the inherent nitrite reductase in a microbe to be modified, or a gene coding for that of some other microbe.

Examples of genes coding for nitrite reductases are the nirD and nirB genes for enzyme (EC 1093349888125_{—}0.4) employing NAD(P)H as an electron donor and the nrfABCDEFG gene (EC 1.7.2.2) or nirA gene (EC 1.7.7.1) for enzyme employing ferredoxin as an electron donor. However, any gene coding for a nitrite reductase that can be expressed in a host may be employed. The gene coding for nitrite reductase may be a native gene or may be a gene derived from some other organism. A portion of the gene may be deleted so long as the gene has nitrite reductase activity. The following sequences, which have already been disclosed in GenBank or the like, may be employed as the gene coding for nitrite reductase.

The following genes and microbes are examples of genes and microbes having nitrite reductase employing NAD(P)H as an electron donor (EC: 1093349888125_{—}1.4), and the protein coded for by the gene may be employed as the nitrite reductase of the present invention.
E. coli
GenBank Accession Number: AAC76390 (nirB) nitrite reductase (NAD(P)H) subunit [gi: 1789765] (SEQ ID NO: 9 (nucleotide nos. 1 to 2544), amino acid SEQ ID NO: 10)
GenBank Accession Number: AAC76391 (nirD) nitrite reductase (NAD(P)H) subunit [gi: 1789766] (SEQ ID NO: 11 (SEQ ID NO: 9: nucleotide sequence nos. 2,541 to 2,864), amino acid SEQ ID NO: 12)
Salmonella typhimurium CT18 GB:
GenBank Accession Number: CAD08139 (nirB) nitrite reductase (NAD(P)H) large subunit [gi: 16505117]
GenBank Accession Number: CAD08138 (nirD) nitrite reductase (NAD(P)H) small subunit [gi: 16505116]
Salmonella typhimurium GB:
GenBank Accession Number: AAL22336 (nirB) nitrite reductase large subunit [gi: 16422031]
GenBank Accession Number: AAL22337 (nirD) nitrite reductase small subunit [gi: 16422032]
Shigella flexneri GB:
GenBank Accession Number: AAP19331 (nirB) nitrite reductase (NAD(P)H) subunit [gi: 30043611] AAN44848 (nirD) nitrite reductase (NAD(P)H) subunit [gi: 24053832]
Bacillus subtilis GB:
GenBank Accession Number: CAB12123 (nasE) assimilatory nitrite reductase (subunit) [gi: 2632615]
GenBank Accession Number: CAB12124 (nasD) assimilatory nitrite reductase (subunit) [gi: 2632616]
GenBank Accession Number: CAB12126 (nasB) assimilatory nitrite reductase (electron transfer subunit) [gi: 2632618]

Desirably, the nirD or nirB gene of E. coli or the nasD, nasE, or nasB gene of Bacillus subtilis is employed. Many of the genes coding for nitrite reductase that are denoted by EC 1.7.1.4 have an operon structure. The protein that nirB codes for constitutes a large subunit of nitrite reductase and the protein that nirD codes for constitutes a small subunit of nitrite reductase. The nirB gene corresponds to nucleotide numbers 1 to 2544 in SEQ ID NO: 9 and the nirD gene corresponds to nucleotide sequence numbers 2541 to 2864 (SEQ ID NO: 11) in SEQ ID NO: 9.

Examples of genes or microbes having enzymes employing ferredoxin as an electron donor (EC 1.7.2.2) are given below. These genes are thought to code for various subunits of nitrite reductases, and the proteins coded for by the genes can be used as the nitrite reductase of the present invention (Mol. Microbiol. April 1994: 12(1): 153-63)(nrfABCDEFG).
E. coli nrf operon (SEQ ID NO: 13)
AAC77040 (nrfA) periplasmic cytochrome [gi: 1790506] (SEQ ID NO: 13, nucleotide nos. 303-1739; amino acid SEQ ID NO: 14)
AAC77041 (nrfB) a penta-haeme cytochrome c [gi: 1790507] (SEQ ID NO: 13, nucleotide nos. 1784-2350; amino acid SEQ ID NO: 15)
AAC77042 (nrfC) Fe—S centers [gi: 2367345] (SEQ ID NO: 13, nucleotide nos. 2347-3018; amino acid SEQ ID NO: 16)
AAC77043 (nrfD) transmembrane protein; [gi: 1790509] (SEQ ID NO: 13, nucleotide nos 3015-3971; amino acid SEQ ID NO: 17)
AAD13457 (nrfE) possible assembly function; [gi: 1790511] (SEQ ID NO: 13, nucleotide nos 4051-5709; amino acid SEQ ID NO: 18)
AAD13458 (nrfF) involved in attachment of haem c to cytochrome c552 [gi: 1790512] (SEQ ID NO: 13, nucleotide nos. 5702-6085; amino acid SEQ ID NO: 19)
AAD13459 (nrfG) [gi: 1790513] (SEQ ID NO: 13, nucleotide nos 6082-6678; amino acid SEQ ID NO: 20)

Examples of genes or microbes having enzymes employing ferredoxin as electron donor (EC 1.7.7.1) are given below. The proteins coded for by the genes can be used as the nitrite reductase of the present invention.
(nirA)
Corynebacterium glutamicum NP_{—}602008 (nirA) putative nitrite reductase [gi: 19554006] (SEQ ID NO: 21)
Mycobacterium tuberculosis CAA17319 (nirA) possible oxidoreductase [gi: 16505265]
Streptomyces coelicolor CAC33947 (nirA) putative nitrite/sulphite reductase [gi: 13276829]
Pyrobaculum aerophilum AAL64294 (nirA) ferredoxin-nitrite reductase [gi: 18160980]
A homologue of the nirA gene of Corynebacterium glutamicum is employed with preference.

The genes coding for these nitrite reductases can be obtained by the methods described below. For example, they can be obtained by polymerase chain reaction (PCR: see White, T. J. et al., Trends Genet. 5, 185 (1989)) employing E. coli chromosomal DNA as template. The oligonucleotides having the nucleotide sequences shown in SEQ ID NOS: 1 and 2 are examples of amplification primers for the nirBD coding for the nitrite reductase of bacteria of the genus Escherichia. Examples of sources of such chromosomal DNA are, when obtaining nirBD and nrfABCDEFG, wild-type strains of E. coli such as strain W3110 (ATCC39936) and strain MG1655, and a wild-type strain 168 of Bacillus subtilis. When obtaining nirA homologues, an example is Corynebacterium glutamicum ATCC13869.

Strains W3110, MG1655, 168, and ATCC13869 are available from the American Type Culture Collection, 10801 University Boulevard, Manassas, Va. 20110-2209, USA.

So long as the nitrite reductase activity is not lost, the nitrite reductase of the present invention may contain one or several amino acid substitutions, deletions, insertions, additions, or inversions at one or several sites. Here, the term “several” varies with the type and position of the amino acid residues within the three-dimensional structure of the protein, but specifically refers to from 2 to 30, preferably from 2 to 20, and more preferably from 2 to 10 amino acids.

DNA coding for substantially the same protein as the above-described nitrite reductases can be obtained by, for example, modifying the coding region of the gene for a nitrite reductase by a site-specific mutagenesis method so that the amino acid sequence denoted by SEQ ID NO: 10, 12, 14 to 20, or 22 contains from 1 to 30 amino acid substitutions, deletions, additions, or inversions. DNA modified in this manner may also be obtained by conventional mutation treatments. Examples of mutation treatments are methods by which a DNA is treated in vitro with hydroxylamine or the like, and methods by which a microbe such as a bacterium of the genus Escherichia having a DNA prior to mutation treatment is treated with a conventionally employed mutating agent such as ultraviolet radiation, N-methyl-N′-nitro-N-nitrosoguanidine (NTG), or EMS.

Mutation of the gene coding for the above nitrite reductase may be a conservative mutation in which the activity of the nitrite reductase is conserved. The above substitution is a change in which one or more residues in the amino acid sequence are removed and another residue is inserted at that site. Examples of the substitution of one or more original amino acids of a nitrite reductase with one or more amino acids in what is considered to be a conservative substitution are: substituting Ser or Thr for Ala; substituting Gln, His, or Lys for Arg; substituting Glu, Gln, Lys, His, or Asp for Asn; substituting Asn, Glu, or Gln for Asp; substituting Ser or Ala for Cys; substituting Asn, Glu, Lys, His, Asp, or Arg for Gln; substituting Asn, Gln, Lys, or Asp for Glu; substituting Pro for Gly; substituting Asn, Lys, Gln, Arg, or Tyr for His; substituting Leu, Met, Val, or Phe for Ile, substituting Ile, Met, Val, or Phe for Leu; substituting Asn, Glu, Gln, His, or Arg for Lys, substituting Ile, Leu, Val, or Phe for Met; substituting Trp, Tyr, Met, Ile, or Leu for Phe; substituting Thr or Ala for Ser; substituting Ser or Ala for Thr; substituting Phe or Tyr for Trp, substituting His, Phe, or Trp for Tyr; and substituting Met, Ile, or Leu for Val.

DNA having the mutation as set forth above is expressed in a suitable cell and the activity of the expression product is tested to obtain a DNA coding for a protein substantially identical to the nitrite reductase.

The gene coding for the nitrite reductase employed in the present invention includes DNA coding for a protein having nitrite reductase activity that hybridizes under stringent conditions with a nucleotide sequence of SEQ ID NOS: 9, 13, or 21, or with a probe prepared from the nucleotide sequence. The term “stringent conditions” as used herein refers to conditions under which a specific hybrid forms and nonspecific hybrids do not form. The reduction of such conditions to numerical values is difficult, but as an example, they are conditions under which DNA strands with a high degree of homology, for example, greater than or equal to 80 percent, preferably greater than or equal to 90 percent, and more preferably, greater than or equal to 95 percent, hybridize, and DNA strands with a lower degree of homology do not hybridize. As a further example, they are the usual Southern hybridization washing once or preferably 2-3 times under 60° C. at a salt concentration corresponding to 1×SSC, 0.1 percent SDS, preferably 0.1×SSC, 0.1 percent SDS.

A portion of the nucleotide sequence of SEQ ID NO: 9, 13, or 21 can be employed as probe, for example. Such a probe can be prepared employing an oligonucleotide prepared based on the nucleotide sequence of SEQ ID NO: 9, 13, or 21 as primer and conducting PCR with a DNA fragment containing the nucleotide sequence of SEQ ID NO: 1 or 3 as template. When a DNA fragment of about 300 bp in length is employed as probe, an example of the hybridization washing conditions is 50° C., 2×SSC, and 0.1 percent SDS.

A specific example of a DNA coding for a protein substantially identical to a nitrite reductase is a DNA preferably having a degree of homology of greater than or equal to 70 percent, preferably greater than or equal to 80 percent, more preferably greater than or equal to 90 percent, and still more preferably greater than or equal to 95 percent with the amino acid sequence given by SEQ ID NO: 10, 12, 14 to 20, or 22, as well as coding for a protein having activity equivalent to that of a nitrite reductase. Any DNA having nitrite reductase activity will suffice.

Genes coding for nitrite reductase of other microbes may be obtained by PCR or the like from the chromosomal DNA of the microbe in the same manner as a well-known method used to obtain a gene.

Chromosomal DNA may be prepared by the method of Saito and Miura (see H. Saito and K. Miura, Biochem. Biophys. Acta, 72, 619 (1963), Book of Biological Experiments, compiled by the Japan Bioengineering Society, pp. 97-98, Baifukan, 1992) from microbes that are DNA donors.

The obtained gene is inserted into vector DNA that can be autonomously replicated in E. coli and/or the cell of the targeted microbe to prepare recombinant DNA. When this is incorporated into E. coli, the subsequent operations are facilitated. Examples of vectors that can be autonomously replicated in E. coli are pSTV29, pUC19, pUC 18, pHSG299, pHSG399, pHSG398, RSF1010, pBR322, pACYC184, and pMW219. The use of an expression vector with a His-Tag or the like to permit purification by large scale expression of a protein in the cell is also possible.

To ligate the obtained gene with a vector functioning in the target cell to prepare recombinant DNA, it suffices to cleave the vector with a restriction enzyme which recognizes the same sequence as the terminal of the above gene and employ a ligase such as T4 DNA ligase to bind the gene to the vector.

Nitrite reductase activity is enhanced by enhancing expression of the gene coding for the protein constituting the nitrite reductase, as set forth above. The level of expression of a single gene is enhanced by increasing the number of copies of the gene in the manner set forth above, for example. By way of example, it suffices to link the above gene fragment to a vector functioning within a microbe, preferably a multicopy-type vector, to prepare recombinant DNA, and introduce this into a host to transform the host.

Transformation methods that have been reported thus far may be employed to introduce the recombinant DNA into a microbe. For example, as reported for E. coli strain K-12, there exists the method of treating a recipient microbial cell with calcium chloride to increase DNA permeability (Mandel, M. and Higa, A., J. Mol. Biol., 53, 159 (1970)), and as reported for Bacillus subtilis, there exists the method of preparing competent cells from cells in the growth stage to introduce DNA (Duncan, C. H., Wilson, G. A., and Young, F. E., Gene, 1, 153 (1977)). Alternatively, it is possible to apply the method, known for Bacillus subtilis, Actinomyces, and yeast, of converting the cells of a DNA recipient microbe into protoplast or spheroplast form, which readily incorporate recombinant DNA, and introducing the recombinant DNA into a DNA recipient microbe (Chang, S. and Choen, S. N., Molec. Gen. Genet., 168, 111 (1979)); Bibb, M. J., Ward, J. M. and Hopwood, O. A., Nature, 274, 398 (1978); Hinnen, A., Hicks, J. B., and Fink, G. R., Proce. Natl. Acad. Sci. USA, 75 1929 (1978)). Microbe transformation is also possible by the electric pulse method (Japanese Patent Application Publication No. Heisei 2-207791).

The number of copies of a gene can also be increased by introducing multiple copies of a gene onto the chromosomal DNA of a microbe. Multiple copies of a gene can be introduced onto the chromosomal DNA of a microbe by employing as target a sequence which are present on chromosomal DNA in multiple copies, and conducting homologous recombination. Repetitive DNA, inverted repeats present at the end of transposon, and the like can be employed as sequences which are present on chromosomal DNA in multiple copies. Alternatively, as disclosed in Japanese Patent Application Publication No. Heisei 2-109985, a target gene can be loaded onto a transposon and transferred to introduce multiple copies onto chromosomal DNA.

In addition to the above-described genetic amplification, nitrite reductase activity can also be enhanced by substituting an expression control sequence, such as a promoter, coding for a nitrite reductase on chromosomal DNA or a plasmid with a stronger expression control sequence. For example, lac promoter, tac promoter, trp promoter, and trc promoter are known as strong promoters. Furthermore, as disclosed in International Application Publication No. WO00/18935, a substitution of several nucleotides can be introduced into a promoter region of a gene to render it stronger. These promoter substitutions or modifications enhance the expression of the gene coding for the nitrite reductase, enhancing nitrite reductase activity. These substitutions and modifications in the expression control sequence may also be combined with an increase in the number of copies of the gene.

The expression of many genes coding for nitrite reductases is regulated under aerobic conditions. By substituting the promoter upstream from the gene coding for a nitrite reductase with a constitutively expressed strong promoter that is not regulated by aerobic conditions, it is possible to increase the level of expression independently of aeration conditions. For example, the substitution of the control region with a tac promoter or the like is conceivable. Furthermore, the incorporation of site-specific mutations so that the control region no longer functions permits an increase in the level of expression that is independent of ventilation conditions.

The usual microbe culturing methods, such as liquid culturing and solid culturing, can be employed to culture a microbe. For reasons of economy, methods of liquid culturing under aerobic conditions are desirable. Examples are the shaking culture method and the aerated stirring culture method under aerobic conditions. The expression of genes coding for nitrate reductase is often regulated under aerobic conditions. When expression is regulated, the method of avoiding contact with air using the paraffin method under anaerobic conditions, the method of adding a reducing agent to the medium, and the method of backfilling a sealed container with nitrogen gas may be employed.

Any culture components that allow the strain being used in the present invention to proliferate may be employed. For example, a culture containing a carbon source in the form of an assimilatable carbon compound, or a substance containing such a compound, can be employed; glucose, starches, starch hydrolysates such as liquid starch, and sugars such as molasses may be employed singly or in combination. As a further example, a nitrogen source in the form of an assimilatable compound, or a substance containing such a compound, can be employed; organic nitrogen-containing compounds including various amino acids, corn steep liquor, malt extract, peptones, soybean meal, defatted soybean meal, and inorganic nitrogen compounds including ammonium chloride, ammonium sulfate, and other ammonium salts may be employed singly or in combination. Still further, various organic and inorganic substances, as well as materials containing the same, that are necessary for the growth of microbes and the production of enzymes may be added. These include salts such as phosphates, magnesium salt, calcium salt, and manganese salt, as well as vitamins and yeast extracts.

The microbe-derived nitrite reductase of the present invention may be a nitrite reductase that is purified from the culture solution of a microbe, for example. Nitrite reductase may be purified by ammonium sulfate precipitation, column chromatography, ethanol precipitation, or the like. So long as the microbe of the microbe-derived nitrite reductase of the present invention exhibits nitrite reduction activity, it may be provided in any of a variety of forms. For example, it may be derived from the culture solution of the microbe by drying the microbe mass itself, be in the form of a microbe culture solution product obtained by treatment with acetone or the like, or be in the form of culture solution containing the microbe.

The nitrite reductase of the present invention may be prepared by culturing the above-described microbe in a culture medium and, during a period where protein purification is easy, such as during the exponential growth phase, purifying the enzyme by ammonium sulfate precipitation, column chromatography, ethanol precipitation, or the like. The unrefined culture solution in liquid form or the dried microbe mass may be employed as a methane production reducer containing nitrite reductase. The enzyme or enzyme solution employed in the present invention can be produced using culture components consistent with the strain employed and the enzyme being targeted. When employed in an enzyme solution, it is effective to continue treatment to obtain a suitably concentrated form.

Enhanced nitrite reductase activity in the rumen can be confirmed using an artificial rumen system (T. Hino et al., J. Gen. Appl. Microbiol., 39, 35-45 (1993) or confirmed in vivo by actual oral administration to a ruminant. The in vitro rumen simulation technique (RUSITEC), described by Czerkawski and Breckenridge (1977) can be used as an artificial rumen system (product of Sunshinkogyo).

The methane production reducer of the present invention may be administered to ruminants in a variety of forms, such as powders, grains, and tablets. Excipients, extenders, and the like may be suitably added as needed. The proportion of the nitrite reductase of the present invention in the methane production reducer may be determined based on the goal for which it is being used, or the like. When protein purity is high or specific activity is high, the nitrite reductase is administered in a low proportion, and when administering the culture medium itself or when specific activity is low, the nitrate reductase is administered in a high proportion. Administration may be directly into the rumen fluid of a ruminant, or a method of oral administration may be employed, for example.

The methane production reducer of the present invention can be administered at any time when feed is present within the rumen; there is no specific limitation. However, since it is desirable for the methane production reducer to be present in the rumen during methane generation, it is desirable for the methane production reducer to be administered immediately before or simultaneously with feed administration. Particularly effective administration is possible by blending the methane production reducer into the feed.

The administration amount of methane production reducer of the present invention is not specifically limited. For example, administration level of nitrite reductase which enhances nitrite reduction activity within the rumen may be 0.1 mM or more of the nitrite reduction activity per hour per volume (L) of rumen (0.1 mM/h/L or more), preferably 0.2 mM/h/L or more. The volume of the rumen can be converted into body weight based on the average volume of the rumen and the average body weight. Suitable adjustment is made for the microbe being employed and the animal receiving the administration.

The feed composition for ruminants of the present invention is a feed composition in which the above-described methane production reducer is added to a conventional feed composition. The blending proportion of the methane production reducer in the feed composition is normally from 0.1 to 10 weight percent, preferably from 1 to 5 weight percent. The conventional feed composition for ruminants is not specifically limited; a commercial product may be employed without alteration, or, as needed, suitable silage or hay may be added to a commercial product. An example of a bovine feed composition is 40 percent corn silage, 14 percent Sudan hay or alfalfa hay cubes, and 46 percent of a commercial blend of feed. An example of a commercial blend of feed contains not less than 16 percent of crude protein and not less than 71 percent digestible total nutrients.

An example of an ovine feed composition contains 85 percent timothy hay and 15 percent of a commercial blend of feed. In the present invention, the feed composition in which a methane production reducer has been blended may be freely given to the ruminant. It may also be given for extended periods.

Administering the feed composition containing a methane production reducer of the present invention reduce or inhibit methane production in the rumen of ruminants and enhances feed efficiency. Furthermore, the growth of ruminants is enhanced and nitrite intoxication of ruminants can be prevented.

The present invention is specifically described below through Examples. However, the present invention is not limited to the Examples given below. To a system in which nitrites and nitrates had been suitably added to rumen fluid in vitro, E. coli with enhanced nitrate reduction activity or a wild-type strain E. coli was added to show how nitrites were rapidly reduced and the generation of methane was reduced.

### EXAMPLE 1

Construction of Eshcerichia Bacterium which has an Enhanced Nitrite Reduction Activity

<Construction of E. coli nir-Ptac Strain which has an Enhanced Nitrite Reduction Activity>

E. coli nir-Ptac strain has nitrite reductase gene nirBD, the native promoter of which is replaced with tac promoter. This strain was constructed as follows: a DNA fragment containing nirBD genes was amplified by PCR with Pyrobest DNA polymerase (Takara Shuzo Co., Ltd., Shiga, Japan), using W3110 genomes as a template, and ni5 (SEQ ID NO: 1/TCA GCC GTC ACC GTC AGC ATA ACA C) and nic4.1 (SEQ ID NO: 2/CCG ACA GGC GTG CAA TGC GCG CAG C) as primers. PCR was conducted with 30 cycles of 10 seconds at 98° C., 30 seconds at 59° C and 3.5 minutes at 72° C. amplified fragment was inserted into the SmaI site of pHSG398 vector (Takara Bio Co., Ltd.) and checked to have no PCR error. The constructed plasmid was designated pNIRBD. Plasmid pMW219-nirBD was constructed by cutting nirBD genes off pNIRBD with KpnI and XbaI, and inserting the nirBD genes into the pMW219 (Nippon Gene) at KpnI and XbaI sites. This plasmid has nirBD operon in the same direction as lacZ gene.

Another PCR with Pyrobest DNA polymerase (Takara Bio Co., Ltd.) was performed using pMW219-nirBD as a template, and nirex5 (SEQ ID NO: 3/AAA AGA ATT CGA GGC AAA AAT GAG CAA AGT) and nirex3 (SEQ ID NO: 4/CCC CAA GCT TCA TGC AAA AAG GGG AGG CAT) as primers. PCR was conducted with 30 cycles of 10 seconds at 98° C., 30 seconds at 57° C and 3.5 minutes at 72° C. The amplified fragment was cut off with EcoRI and HindIII and inserted into the pKK223-3 expression vector (Amersham Pharmacia Biotech) at the EcoRI and HindIII sites. The inserted fragment was checked and found to have no PCR error. The constructed plasmid was designated pKK-nirEx.

A DNA fragment with the tac promoter used for gene expression studies in E. coli (Xue et al., 1996, Temperature-regulated expression of the tac/Lacl system for overproduction of a fingal xylanase in Escherichia coli Appl. Microbiol. Biotechnol. 45, 120-126) and a 5′ portion of the nirBD genes was amplified by PCR with Pyrobest DNA polymerase (Takara Bio Co., Ltd.), using pKK-nirEx as a template, and pKK-c200 (SEQ ID NO: 5/CGG GGT ACC TTC TGG CGT CAG GCA GCC AT) and NI′-c2035 (SEQ ID NO: 6/ACA TGC ATG CCG TCT ACG CCC AGC AGT TTC) as primers. PCR was conducted with 30 cycles of 10 seconds at 98° C., 30 seconds at 57° C. and 1 minutes at 72° C. The fragment amplified was digested with KpnI and SphI and designated frag. 1.

A DNA fragment containing the upstream region of nirBD was amplified by PCR with Pyrobest DNA polymerase (Takara Bio Co., Ltd.), using W3110 genome as a template, and NI′-1 (SEQ ID NO: 7/CGG AAT TCG TAT GAA GGG CGT CAG CGC G) and NI′-c925 (SEQ ID NO: 8/CGG GGT ACC TTC TTA AGT CAC GGA ATT GT) as primers. PCR was conducted with 30 cycles of 10 seconds at 98° C., 30 seconds at 57° C. and 1 minute at 72° C. The fragment amplified was digested with KpnI and EcoRI and designated frag.2.

pHSG299 vector (Takara Bio Co., Ltd.) was digested with EcoRI and SphI and ligated with frag.1 and frag.2 constructed above. A clone which has one copy of frag.1 located at EcoRI end and one copy of frag.2 located at SphI end of the digested pHSG299 was chosen. The inserted fragment was checked to have no PCR error. The constructed plasmid was designated pHSG-nir-Ptac.

The inserted fragment of pHSG-nir-Ptac was cut out with HindIII and ligated into HindIII sites of temperature-sensitive vector, pMAN997 (International publication pamphlet WO99/03988). The resulting plasmid was designated pMAN-nir-Ptac. Escherichia coli strain W3110 was transformed with pMAN-nir-Ptac according to a method of C. T. Chung et al. and cultured at 30° C. on LB+ampicillin plate. Colonies which appeared on the plate were selected and cultured overnight at 30° C. The obtained culture was diluted to 10⁻³, spread onto LB+ampicillin plate and cultured at 42° C. Colonies which appeared on the plate were selected and spread onto LB+ampicillin plate and cultured at 30° C. Colonies which appeared on a ⅛ part of the plate were suspended in 2 ml of LB medium and cultured with shaking for 4-5 hours at 42° C. The obtained culture was diluted to 10⁻⁵ and spread onto LB plate. A few hundred of the obtained colonies were seeded onto an LB plate and an LB+ampicillin plate. Ampicillin-sensitive strains were selected by comparing growth on the both plates. The ampicillin-sensitive strains were subject to colony PCR for selecting clones with the promoter region of nirBD replaced by tac promoter through the twice recombination between genome and plasmid. The selected clone was checked to have no vector region on its genome and designated nir-Ptac.

| | |
|---|---|
| ni5: | |
| tca gcc gtc acc gtc agc ata aca c | (SEQ ID NO:1) |
| nic4.1: | |
| ccg aca ggc gtg caa tgc gcg cag c | (SEQ ID NO:2) |
| nirex5: | |
| aaa aga att cga ggc aaa aat gag caa agt | (SEQ ID NO:3) |
| nirex3: | |
| ccc caa gct tca tgc aaa aag ggg agg cat | (SEQ ID NO:4) |
| pKK-c200: | |
| cgg ggt acc ttc tgg cgt cag gca gcc at | (SEQ ID NO:5) |
| NI′-c2035: | |
| aca tgc atg ccg tct acg ccc agc agt ttc | (SEQ ID NO:6) |
| NI′-1: | |
| cgg aat tcg tat gaa ggg cgt cag cgc g | (SEQ ID NO:7) |
| NI′-c925: | |
| cgg ggt acc ttc tta agt cac gga att gt | (SEQ ID NO:8) |

<Analysis of Nitrate and Nitrite Concentration>

Nitrate and nitrite concentration in culture supernatant was roughly estimated using NO₂⁻ test paper (Merck: Merckoquant Nitrite Test 1. 10022.0001, 1.10007.0001) and NO₃⁻ test paper (Merck: Merckoquant Nitrate Test 1. 10020.0001), respectively. Then, the sample was diluted to fit the nitrate and nitrite concentration between 0 and 100 μM which is the measurement range of the nitrate/nitrite Assay Kit-C (Colorimetric, Dojindo) and subject to the more accurate concentration measurement with the kit. The absorption coefficient was measured using the microplate reader SPECTRA MAX 190 (Molecular Devices).

### EXAMPLE 2

Effect in in vitro Rumen System

<Materials and Experimental Tools>

The in vitro continuous incubation systems consisting of four 1000-ml fermenter vessels equipped with a buffer and solid feed input, a thermister probe, an input for nitrogen gas, magnetically stirring and near-infrared CH₄ and CO₂ analyzer (Takasugi Seisakusho Co. Ltd. Tokyo, Japan) were used in the Examples. Two nonlactating Holstein cows (average 800 kg BW) used as donor cows were fitted with ruminal fistulae according to surgical procedures approved by the Institutional Animal Care and Use Committee of Obihiro University of Agriculture and Veterinary Medicine, and was housed in a tie-stall barn. The cows received a basal diet of hay (DM (dry matter): 87.33%, OM (organic matter): 98.98%, CP (crude protein): 14%, ADF (acid detergent fiber): 38.84%, NDF (neutral detergent fiber): 73.26%, ADL (acid detergent lignin): 4.10%, GE (gross energy): 4.45 Mcal) at maintenance level (55 g DM kg^{-0.75} BW/day) in two equal portions at 08:00 and 17:00 h with free access to ad libitum water and block sodium chloride (Fe: 1232, Cu: 150, Co: 25, Zn: 500, I: 50, Se: 15 and Na: 382 mg/kg). Rumen fluid was collected from the cows before morning feeding and strained immediately through woven nylon cloth into Erlenmeyer flask with O₂-free headspace. One volume of strained rumen fluid (400 ml) was mixed with one volume of autoclaved buffer solution (400 ml) (Takahashi, J. et al., Inhibitory effects of sulphur compounds, copper and tungsten on nitrate reduction by mixed rumen micro-organisms. Br. J. Nutr. 61, 741-748). All incubations were carried out anaerobically at 39° C. for 24 h with the addition of 10 g of the ground (1-mm screen) diet described above as substrate and were performed in quadruplicate. Two vessels per treatment were randomly assigned. Anaerobic conditions were achieved by continuous infusion of N₂ at a rate of 20 ml/min. Maintaining the fermenters' temperature at 39° C. and mixing of their contents were achieved by using the Takasugi Seisakusho-Culture fermenter base units (Takasugi Seisakusho Co. Ltd. Tokyo, Japan).

<Supplement, Inoculation and Cultures of E. coli W3110 or E. coli nir-Ptac>

For the purpose of studying the effects of nitrate, nitrite, E. coli W3110, and E. coli nir-Ptac on in vitro ruminal methanogenesis by the ruminal bacteria, the incubation vessels were supplemented with nitrate (NaNO₃, 5 or 10 mM) or nitrite (NaNO₂, 1 or 2 mM) and inoculated with cultured E. coli W3110 or E. coli nir-Ptac. For the purpose of studying the effect of E. coli W3110 or E. coli nir-Ptac on in vitro ruminal nitrate and nitrite reduction by ruminal bacteria, cultured E. coli W3110 or E. coli nir-Ptac were inoculated into separate incubation vessels supplemented with nitrate (NaNO₃, 5 or 10 mM) or nitrite (NaNO₂, 1 or 2 mM). The cell amounts of E. coli W3110 or E. coli nir-Ptac inoculated into each incubation vessel were calculated as indicated in the below equation. The control culture was incubated without added nitrate and nitrite, and inoculated E. coli W3110 or E. coli nir-Ptac.

E. coli nir-Ptac was Constructed According to Example 1.

E. coli W3110 or E. coli nir-Ptac inoculum were prepared by culturing the cells in the Luria-Bertani (LB) broth agar at 37° C. for 10 hr. One-eight of E. coli W3110 or E. coli nir-Ptac cells on the agar plate were collected and put into flasks with 50 ml of LB broth (10 g of tryptone, 5 g of yeast extract, 10 g of NaCl per liter) and incubated for a further 12 hr at 37° C. with constant shaking (150 rev. min⁻¹), respectively. Stationary phase cells were harvested with centrifugation (5000×g, 5 min, 4° C.). Cell pellets of E. coli W3110 or E. coli nir-Ptac were suspended in 25 ml of sterile buffer solution (pH 6.8) (McDaugall, 1948, Studies on ruminant saliva.1. The composition and output of sheep's saliva. Biochem. J. 43, 99-109) and re-suspended in 5 ml of sterile buffer solution, respectively. The amount of the E. coli W3110 or E. coli nir-Ptac to be inoculated into each fermenter was calculated according to the equation:
E=(A)×(B)/(C)×(D)

Where E: the amount of the E. coli cells to be inoculated (ml).
A: OD₆₆₀ value of solution in fermenter assumed at 2.
B: the solution volume in fermenter (800 ml).
C: OD₆₆₀ value of cultured E. coli W3110 or E. coli nir-Ptac cells diluted 1:200 with buffer solution (McDaugall, 1948 Studies on ruminant saliva.1. The composition and output of sheep's saliva. Biochem. J. 43, 99-109).
D: dilution rate (200)

According to the above calculation, E. coli W3110 or E. coli nir-Ptac cells inoculated into each fermenter ranged from 30.53 to 39.40 ml (approximately 1×10⁹ cfu/ml).

<Analytical Procedures>

The above described materials were put into the in vitro rumen system to start the reaction. Rumen sample was collected at 0, 0.5, 1, 1.5, 2, 4, 6, 8, 10, 12, and 24 h to immediately determine pH and cell growth (OD₆₆₀), and then each sample was stored at −20° C. for later determination of ruminal nitrate, nitrite, ammonia nitrogen and volatile fatty acid. The value of pH in rumen fluid was measured using a pH meter (HM-21P, TOA Electronics Ltd., Tokyo, Japan). Optical density at 660 nm (OD₆₆₀) was used as an index of cell growth of the bacterial population as well as of E. coli W3110 and E. coli nir-Ptac of the culture. Samples were diluted 1:200 with distilled water before measurement of OD₆₆₀ with spectrophotometer (Part No. 100-004, Serial No. 5667-15, Hitachi Ltd., Tokyo, Japan). Nitrate and nitrite concentration in culture supernatant was roughly estimated using NO₂⁻ test paper (Merck: Merckoquant Nitrite Test 1. 10022.0001, 1.10007.0001) and NO₃⁻ test paper (Merck: Merckoquant Nitrate Test 1. 10020.0001), respectively. Then, the sample was diluted to fit the nitrate and nitrite concentration between 0 and 100 μM which is the measurement range of the nitrate/nitrite Assay Kit-C (Colorimetric, Dojindo) and subject to the more accurate concentration measurement with the kit. The absorption coefficient was measured using the microplate reader SPECTRA MAX 190 (Molecular Devices). Amnionia nitrogen concentration was estimated as described previously (Sar et al., 2004 Manipulation of rumen methanogenesis by the combination of nitrate with β 1-4 galacto-oligosaccharides or nisin in sheep. Anim Feed Sci Technol (in press)). The concentrations of VFA were analyzed by gas-liquid chromatography (Shimadzu GC-14A, Kyoto, Japan) equipped with a flame-ionization detector and a-capillary column (ULBON HR-52, 0.53 mm I.D.×30 m 3.0 μm) by using 2-Ethyl-n-butyric acid as the internal standard. Values were calculated automatically using a Chromatopac data processing system (C-R 4A; Shimadzu). Continuous CH₄ and carbon dioxide (CO₂) production rate were automatically measured using an infrared gas analyzer (Takasugi Seisakusho Co. Ltd. Tokyo, Japan), and then those data were taken and pooled into the computer (Windows® XP Professional 1-2 CPU, IMB Corporation) from the analyzer through an interface at 1 min intervals.

<Statistical Analysis>

In vitro continuous incubation in each treatment were performed for 2 days with two replicates per day (n=4). Cumulative CH₄ and CO₂ production were extrapolated by non-linear regression analyses of Bertalanffy model [(CH₄ (ml) or CO₂ (ml)=a+b(1-e^{-ct})³, where a: first CH₄ or CO₂ production, b: second CH₄ or CO₂ production, c: constant CH₄ and CO₂ production rate, t: time (min)) from the time course of CH₄ or CO₂ production for 24 h incubation. Means of results from treatments were analyzed by one-way analysis of variance (ANOVA) using the General Linear Models Procedures of the Statistical Analysis Systems Institute (SAS, 1994). Treatment means were statistically compared with Duncan's multiple-range test. Differences with P<0.05 were considered significant.

<Result>

The result of Example 2 is shown in Table 1.

TABLE 1
Ruminal pH, OD₆₆₀, VFA concentration, NH₃—N concentration, and CH₄ and CO₂ production in in vitro continuous incubation¹

| | | | | Acetate | Propionate | | | | | | |
|---|---|---|---|---|---|---|---|---|---|---|---|
| | | | Total VFA | (A) | (P) | Butyrate | Others² | A/P | NH₃—N | CH₄³ | CO₂³ |
| Treatments | pH | OD₆₆₀ | (mM) | (mM) | (mM) | (mM) | (mM) | ratio | (mg/l) | (ml/min) | (ml/min) |
| Control | 6.90^{4a} | 0.103^{ab} | 39.89^{abc} | 29.37^{abc} | 6.99^{a} | 2.88^{abc} | 0.20^{edc} | 4.15^{bc} | 25.31^{ab} | 0.187^{a} | 0.728^{a} |
| E. coli W3110 | 7.10^{bcd} | 0.150^{cd} | 48.41^{a} | 35.82^{a} | 7.59^{a} | 3.72^{a} | 1.08^{a} | 4.79^{abc} | 67.27^{e} | 0.136^{b} | 0.338^{b} |
| E. coli nir-Ptac | 7.01^{abc} | 0.114^{ab} | 24.49^{e} | 18.53^{e} | 4.36^{bc} | 1.52^{efg} | 0.05^{e} | 4.25^{bc} | 16.49^{a} | 0.127^{b} | 0.467^{ab} |
| Nitrate 5 mM | 7.11^{bcd} | 0.095^{ab} | 36.73^{bcd} | 28.25^{abcd} | 5.27^{b} | 2.60^{bcde} | 0.65^{abc} | 5.15^{ab} | 52.31^{cde} | 0.070^{c} | 0.536^{ab} |
| Nitrate 5 mM + E. coli | 7.04^{abcd} | 0.116^{bc} | 41.60^{ab} | 30.86^{ab} | 7.46^{a} | 2.68^{bcd} | 0.60^{bcd} | 4.14^{bc} | 20.76^{ab} | 0.012^{d} | 0.355^{b} |
| W3110 | | | | | | | | | | | |
| Nitrate 5 mM + E.coli | 7.12^{cd} | 0.161^{d} | 23.19^{e} | 17.68^{e} | 3.39^{c} | 1.67^{defg} | 0.44^{bcde} | 5.12^{abc} | 56.79^{de} | 0.013^{d} | 0.435^{ab} |
| nir-Ptac | | | | | | | | | | | |
| Nitrate 10 mM | 7.17^{d} | 0.105^{ab} | 26.66^{de} | 21.08^{cde} | 4.18^{bc} | 1.38^{fg} | 0.03^{e} | 5.06^{abc} | 60.21^{de} | 0.037^{cd} | 0.385^{b} |
| Nitrate 10 mM + E. coli | 7.08^{bcd} | 0.164^{d} | 46.41^{ab} | 34.81^{a} | 7.46^{a} | 3.44^{ab} | 0.70^{ab} | 4.70^{abc} | 67.46^{e} | 0.010^{d} | 0.331^{b} |
| W3110 | | | | | | | | | | | |
| Nitrate 10 mM + E. coli | 7.12^{bcd} | 0.159^{d} | 28.26^{de} | 20.51^{de} | 5.11^{b} | 1.94^{cdefg} | 0.71^{ab} | 4.04^{c} | 54.41^{cde} | 0.004^{d} | 0.445^{ab} |
| nir-Ptac | | | | | | | | | | | |
| Nitrite 1 mM | 7.08^{bcd} | 0.107^{ab} | 29.05^{de} | 22.21^{cde} | 4.67^{bc} | 2.12^{cdefg} | 0.03^{e} | 4.75^{abc} | 60.92^{de} | 0.133^{b} | 0.721^{a} |
| Nitrite 1 mM + E. coli | 7.09^{bcd} | 0.154^{d} | 29.69^{cde} | 21.84^{cde} | 5.23^{b} | 2.43^{bcdef} | 0.18^{ed} | 4.247^{bc} | 55.12^{cde} | 0.127^{b} | 0.708^{a} |
| W3110 | | | | | | | | | | | |
| Nitrite 1 mM + E.coli | 6.96^{ab} | 0.113^{ab} | 28.74^{de} | 22.06^{cde} | 5.16^{b} | 1.53^{efg} | 0.00^{e} | 4.28^{bc} | 22.18^{ab} | 0.059^{c} | 0.277^{b} |
| nir-Ptac | | | | | | | | | | | |
| Nitrite 2 mM | 7.09^{bcd} | 0.077^{a} | 21.59^{e} | 16.37^{e} | 3.98^{bc} | 1.25^{g} | 0.02^{e} | 4.14^{bc} | 33.71^{abc} | 0.029^{d} | 0.381^{b} |
| Nitrite 2 mM + E. coli | 7.03^{abcd} | 0.149^{cd} | 29.62^{cde} | 23.52^{bcde} | 4.30^{bc} | 1.79^{cdefg} | 0.01^{e} | 5.49^{a} | 42.21^{bcd} | 0.057^{c} | 0.297^{b} |
| W3110 | | | | | | | | | | | |
| Nitrite 2 mM + E.coli | 6.97^{abc} | 0.110^{ab} | 44.83^{ab} | 35.19^{a} | 8.00^{a} | 1.63^{defg} | 0.00^{e} | 4.40^{bc} | 24.57^{ab} | 0.001^{d} | 0.226^{b} |
| nir-Ptac | | | | | | | | | | | |
| SEM | 0.045 | 0.0114 | 3.410 | 2.683 | 0.432 | 0.338 | 0.148 | 0.305 | 7.090 | 0.0122 | 0.0932 |
| P value | 0.009 | 0.0001 | 0.0001 | 0.0001 | 0.0001 | 0.0001 | 0.0001 | 0.034 | 0.0001 | 0.0001 | 0.004 |

¹Values are means of 11 sampling times unless otherwise indicated
²Including valeric, iso-valeric and iso-butyric fatty acid.
³Values are means of 24 h incubation
⁴Means within columns with different superscripts (^{a},^{b},^{c},^{d},^{e},^{f},^{g}) differ significantly (P < 0.05). Each value indicates means of four replications

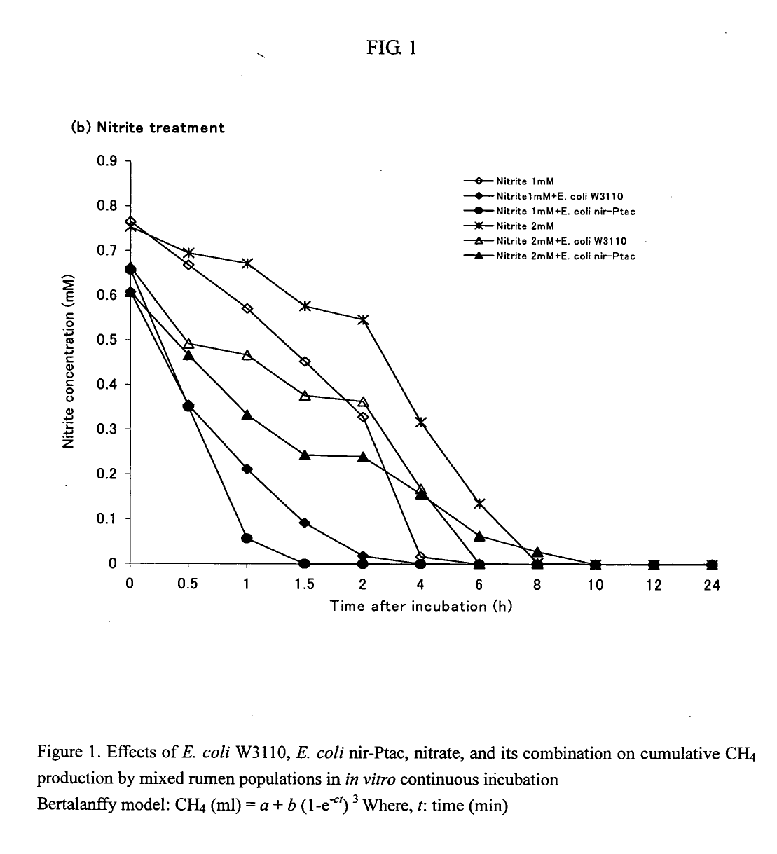

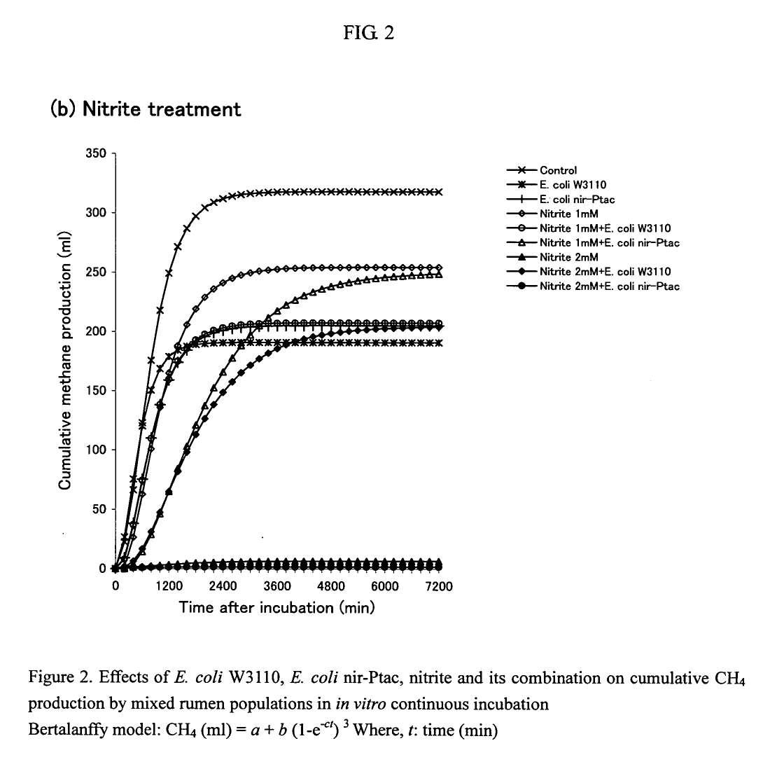

FIG. 1 and FIG. 2 show profiles of in vitro cumulative CH₄ production in cultures of mixed ruminal bacteria treated with nitrate and nitrite, respectively. Cumulative CH₄ production was reduced in all treatments compared to control incubation. When E. coli W3110 was inoculated to 5 or 10 mM-nitrate, reduced cumulative CH₄ production was observed compared to 5 or 10 mM-nitrate without inoculation of the strain. Cumulative CH₄ production was drastically decreased by inoculation of E. coli nir-Ptac to 5 or 10 mM-nitrate and 1 or 2 mM-nitrite compared to 5 or 10 mM-nitrate and 1 or 2 mM-nitrite without inoculation of the strain, respectively.

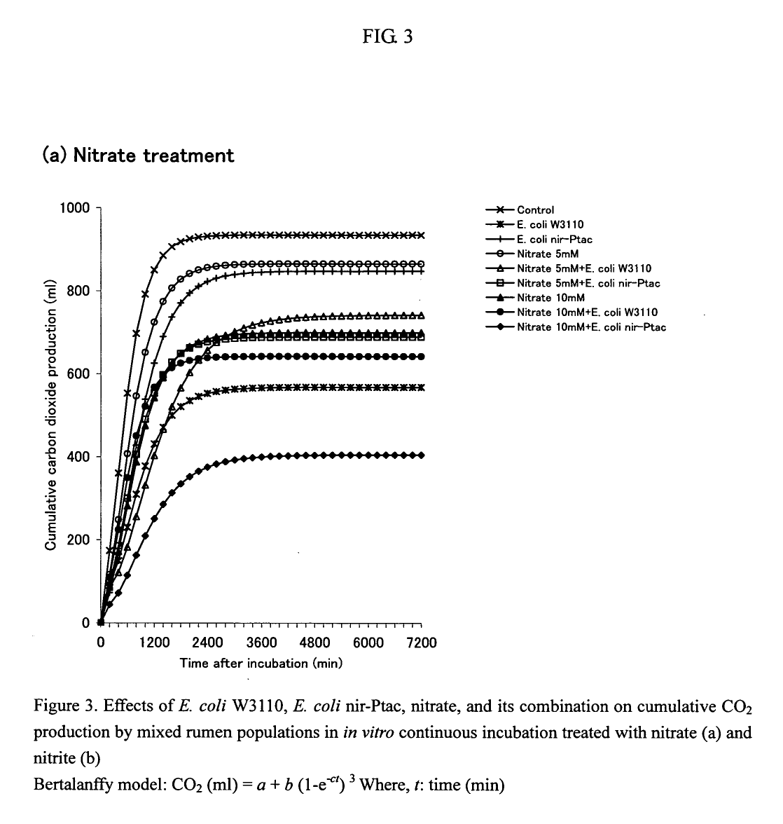

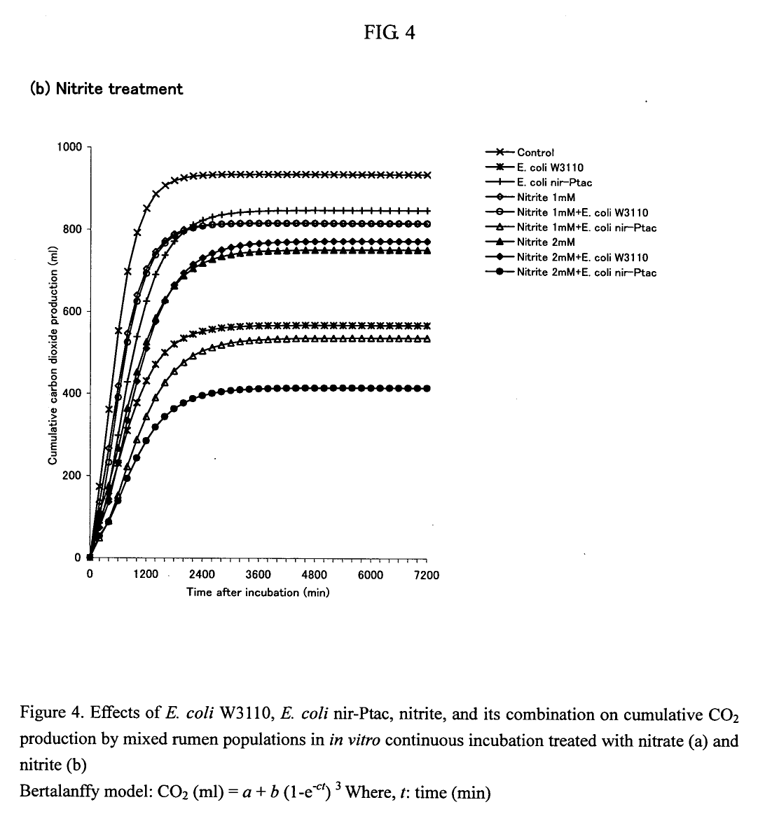

FIG. 3 and FIG. 4 show profiles of in vitro cumulative CO₂ production in cultures of mixed ruminal bacteria treated with nitrate and nitrite, respectively. Reduced cumulative CO₂ production in all treatments was observed compared to control incubation. In treatments of nitrate, the lowest cumulative CO₂ production was obtained in 10 mM-nitrate plus E. coli nir-Ptac. In treatments of nitrite, cumulative CO₂ production was drastically reduced to greatest extents in 2 mM-nitrite plus E. coli nir-Ptac.

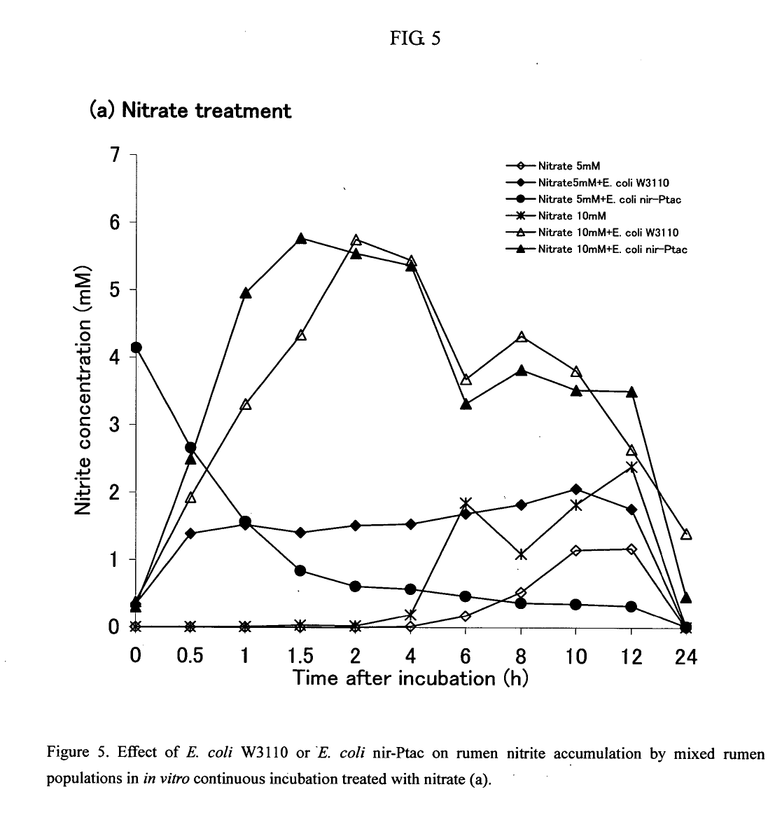

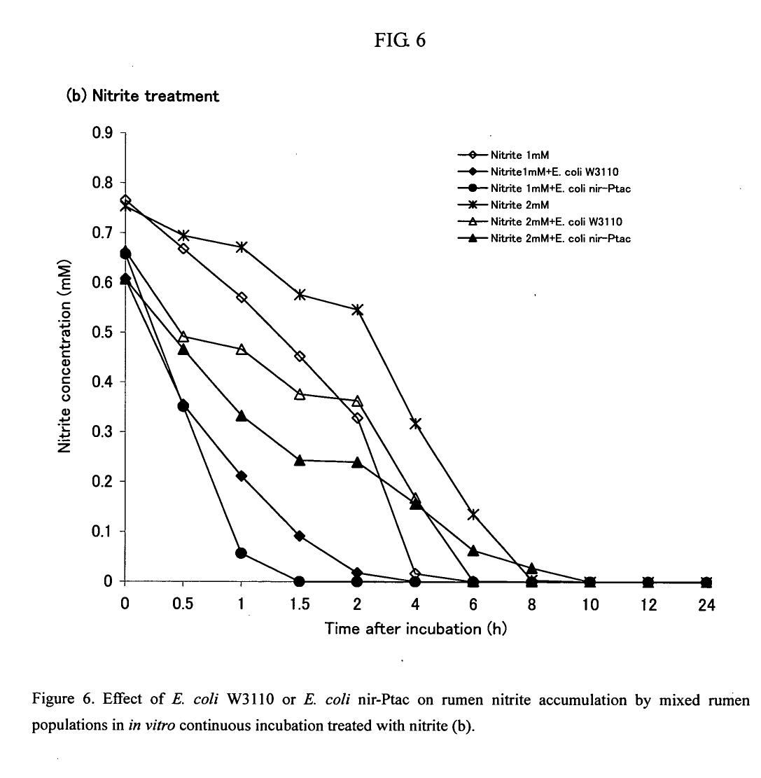

FIG. 5 and FIG. 6 show profiles of ruminal nitrite accumulation in in vitro continuous incubation of mixed ruminal bacteria treated with nitrate and nitrite, respectively. In the treatment of nitrate, an inoculation of E. coli W3110 and E. coli nir-Ptac to mixed rumen population treated with 5 or 10 mM-nitrate resulted in an (P>0.05) increase in ruminal nitrite concentration compared to 5 or 10 mM-nitrate without inoculation of those strains, respectively. In the treatment of nitrite, ruminal nitrite concentration in 1 mM-nitrite plus E. coli W3110 decreased (P<0.01) compared to 1 mM-nitrite without inoculation of the strain, the (P<0.01) greatest values corresponding to 1 mM-nitrite plus E. coli nir-Ptac. Compared to 2 mM-nitrite, the ruminal nitrite concentration was decreased (P<0.01) with 2 mM-nitrite plus E. coli W3110, the greatest (P<0.01) values being found in 2 mM-nitrite plus E. coli nir-Ptac.

The above examples show that wild-type E. coli W3110 and E. coli nir-Ptac reduce the amount of nitrite in rumen, repress negative effect of nitrate and nitrite, and reduce in vitro ruminal methane production.

| | |
|---|---|
| ni5: | |
| tca gcc gtc acc gtc agc ata aca c | (SEQ ID NO:1) |
| nic4.1: | |
| ccg aca ggc gtg caa tgc gcg cag c | (SEQ ID NO:2) |
| nirex5: | |
| aaa aga att cga ggc aaa aat gag caa agt | (SEQ ID NO:3) |
| nirex3: | |
| ccc caa gct tca tgc aaa aag ggg agg cat | (SEQ ID NO:4) |
| pKK-c200: | |
| cgg ggt acc ttc tgg cgt cag gca gcc at | (SEQ ID NO:5) |
| NI′-c2035: | |
| aca tgc atg ccg tct acg ccc agc agt ttc | (SEQ ID NO:6) |
| NI′-1: | |
| cgg aat tcg tat gaa ggg cgt cag cgc g | (SEQ ID NO:7) |
| NI′-c925: | |
| cgg ggt acc ttc tta agt cac gga att gt | (SEQ ID NO:8) |

Escherichia coli NirBD operon NirB gene (SEQ ID NO: 9)

Escherichia coli NirB protein (SEQ ID NO: 10)

Escherichia coli NirD gene (SEQ ID NO: 11)

Escherichia coli NirD protein (SEQ ID NO: 12)

Escherichia coli NrfABCDEFG operon (SEQ ID NO: 13)

Escherichia coli NrfA protein (SEQ ID NO: 14)

Escherichia coli NRfB protein (SEQ ID NO: 15)

Escherichia coli NrfC protein (SEQ ID NO: 16)

Escherichia coli NrfD protein (SEQ ID NO: 17)

Escherichia coli NrfE protein (SEQ ID NO: 18)

Escherichia coli NrfF protein (SEQ ID NO: 19)

Escherichia coli NrfD protein (SEQ ID NO: 20)

Corynebacterium glutamicum NirA gene (SEQ ID NO: 21)

Corynebacterium glutamicum NirA protein (SEQ ID NO: 22)

While the invention has been described in detail with reference to the preferred embodiments thereof, it will be apparent to one skilled in the art that various changes can be made, and equivalents employed, without departing from the scope of the invention. Each of the aforementioned documents, including the foreign priority document JP2004-245705, filed Aug. 25, 2004, are hereby incorporated by reference.

1. A feed composition for ruminants comprising a microbe having nitrite reductase activity, wherein said microbe has been modified so that intracellular nitrite reductase activity is enhanced.

2. The feed composition of claim 1, wherein said microbe is selected from the group consisting of intestinal bacterium, coryneform bacterium, Bacillus bacterium, bacterium belonging to the genus Methylophilus, Actinomyces, ruminal bacteria, and a combination thereof.

3. The feed composition of claim 1, wherein said microbe is Escherichia bacterium.

4. The feed composition of claim 1 wherein said nitrite reductase comprises a protein selected from the group consisting of:
(A) a protein selected from the group consisting of SEQ ID NO: 10, 12, 14 to 20, 22, and combination thereof; and
(B) a protein selected from the group consisting of SEQ ID NO: 10, 12, 14 to 20, 22, and combination thereof, except that 1 to 30 amino acids have been substituted, deleted, inserted, or added, and wherein said protein has nitrite reductase activity.

5. The feed composition of claim 1, wherein said microbe is characterized by enhanced expression of a gene coding for nitrite reductase.

6. The feed composition of claim 5 wherein said gene coding for nitrite reductase is derived from a bacterium of the genus Escherichia, a coryneform bacterium, or a bacterium of the genus Bacillus.

7. The feed composition of claim 6, wherein said gene coding for said nitrite reductase comprises a nucleotide sequence selected from the group consisting of:
(A) a nucleotide sequence selected from the group consisting of SEQ ID NO: 9, 13, and 21; and
(B) a nucleotide sequence which is able to hybrid under stringent conditions with the nucleotide sequence selected from the group consisting of SEQ ID NO: 9, 13, and 21, or with a probe prepared from one of said nucleotide sequences, wherein said gene codes for a protein having nitrite reductase activity.

8. A method of improving the growth of a ruminant by administering the feed composition of claim 1 to said ruminant.

9. The method of claim 8 wherein said microbe has been modified so that intracellular nitrite reductase activity is enhanced.

10. The method of claim 8 wherein said nitrite reductase comprises a protein selected from the group consisting of:
(A) a protein selected from the group consisting of SEQ ID NO: 10, 12, 14 to 20, 22, and combination thereof; and
(B) a protein selected from the group consisting of SEQ ID NO: 10, 12, 14 to 20, 22, and combination thereof, except that 1 to 30 amino acids in have been substituted, deleted, inserted, or added, and wherein said protein has nitrite reductase activity.

11. The method of claim 8 wherein said microbe is characterized by enhanced expression of a gene coding for nitrite reductase.

12. The method of claim 11 wherein said gene coding for nitrite reductase is derived from a bacterium of the genus Escherichia,a coryneform bacterium, or a bacterium of the genus Bacillus.

13. The method of claim 12, wherein said gene coding for said nitrite reductase comprises a nucleotide sequence selected from the group consisting of:
(A) a nucleotide sequence selected from the group consisting of SEQ ID NO: 9, 13, and 21; and
(B) a nucleotide sequence which is able to hybrid under stringent conditions with the nucleotide sequence selected from the group consisting of SEQ ID NO: 9, 13, and 21, or with a probe prepared from one of said nucleotide sequences, wherein said DNA codes for a protein having nitrite reductase activity.

14. A method of preventing nitrite intoxication by administering the feed composition of claim 1 to a ruminant.

15. The method of claim 14 wherein said microbe has been modified so that intracellular nitrite reductase activity is enhanced.

16. The method of claim 15 wherein said nitrite reductase comprises a protein selected from the group consisting of:
(A) a protein selected from the group consisting of SEQ ID NO: 10, 12, 14 to 20, 22, and combination thereof; and
(B) a protein selected from the group consisting of SEQ ID NO: 10, 12, 14 to 20, 22, and combination thereof, except that 1 to 30 amino acids in have been substituted, deleted, inserted, or added, and wherein said protein has nitrite reductase activity.

17. The method of claim 14 wherein said microbe is characterized by enhanced expression of a gene coding for nitrite reductase.

18. A feed composition for ruminants comprising a microbe having nitrite reductase activity, wherein said microbe is selected from the group consisting of intestinal bacteria, coryneform bacteria, Bacillus subtilis, bacteria of the genus Methylophilus, Actinomyces, ruminal bacteria, and a combination thereof.

19. The feed composition of claim 18 wherein said nitrite reductase comprises a protein selected from the group consisting of:
(A) a protein selected from the group consisting of SEQ ID NO: 10, 12, 14 to 20, 22, and combination thereof, and
(B) a protein selected from the group consisting of SEQ ID NO: 10, 12, 14 to 20, 22, and combination thereof, except that 1 to 30 amino acids have been substituted, deleted, inserted, or added, and wherein said protein has nitrite reductase activity.

20. A method of improving the growth of a ruminant by administering the feed composition of claim 18 to said ruminant.

21. A method of preventing nitrite intoxication by administering the feed composition of claim 18 to a ruminant.
